(19) United States
(12) Patent Application Publication
(10) Pub. No.: US 2023/0414663 A1
(43) Pub. Date: Dec. 28, 2023

(54) COMPOSITION BASED ON AUTOLOGOUS PLATELET CONCENTRATES AND A COLOSTRUM ISOLATE MIXTURE OF BIOLOGICAL FACTORS FOR USE IN THE TREATMENT OF CONDITIONS REQUIRING TISSUE REPAIR AND REGENERATION

(71) Applicant: BIOMEDICAL RESEARCH S.R.L., Catania (IT)

(72) Inventor: Giuseppa ZARBO, Catania (IT)

(21) Appl. No.: 18/248,825

(22) PCT Filed: Oct. 15, 2021
(86) PCT No.: PCT/IB2021/059513

§ 371 Date: Apr. 12, 2023

(30) Foreign Application Priority Data
Oct. 15, 2020 (IT) 102020000024385

(51) Int. Cl. A61K 35/19; A61K 35/20; A61K 9/00; A61K 47/36; A61K 47/18; A61P 27/04; A61P 17/02

(52) U.S. Cl. CPC A61K 35/19; A61K 35/20; A61K 9/0014; A61P 17/02; A61K 9/0019; A61K 47/183; A61P 27/04; A61K 47/36

(57) ABSTRACT

Topical or injection compositions for use in the treatment of conditions requiring tissue repair and regeneration in humans and animals are provided. The topical or injection compositions include autologous platelet concentrates and a colostrum isolate mixture of biological factors.

The present invention relates to topical or injection compositions based on autologous platelet concentrates (APCs) and a colostrum isolate mixture of biological factors (containing growth factors, cytokines, chemotactic factors, stem cell stimulating factors, complement proteins C3a/C4a, antibacterial and antiviral factors) for use in the treatment of conditions which require tissue repair and regeneration, in humans and animals.

### TECHNICAL FIELD OF THE INVENTION

Tissue injury healing is a complex process involving a cascade of cellular and molecular events mostly shared by the different tissues of the body. Interestingly, the tissue repair process begins immediately after a traumatic injury and is mediated and controlled by a wide range of cytokines, proteins and growth factors, some of which are released from platelets after activation.

In recent years, the development of platelet-rich preparations has revolutionized the field of regenerative medicine due to the repair capacity of growth factors, released by the platelets themselves, which stimulate and accelerate tissue healing and regeneration.

Autologous platelet concentrates (APCs) are blood components obtained by centrifuging the patient's blood in order to collect the most active components: platelets, fibrin and, in some cases, even leukocytes. The final product has a platelet concentration above the baseline level, consequently it has a higher number of growth factors derived from the platelets themselves. The rationale for the clinical use of such preparations is based on the concept of exploiting the enriched content thereof of several platelet-derived mitogenic growth factors (including platelet-derived growth factor or PDGF, transforming growth factor-β or TGF-β, endothelial growth factor or EGF, insulin-like growth factor-1 or IGF-1, vascular endothelial growth factor or VEGF, fibroblast growth factor or FGF and hepatocyte growth factor or HGF) to stimulate different biological functions, such as chemotaxis, angiogenesis, proliferation and differentiation, so as to promote the healing of hard and soft tissues (FIG. 1).

Brief History of Platelet Concentrates

1. Fibrin Adhesives.

More than 40 years ago these technologies were used to make sealing adhesive agents for the treatment of bleeding, with the function of blocking blood loss. Subsequently other molecules involved in the coagulation process were combined with such fibrin preparations to improve the adhesive properties thereof. These preparations, termed “platelet-fibrinogen-thrombin mixtures”, have been used successfully in ophthalmology, general surgery, and neurosurgery. Other authors have defined them as “gel foam”.

Interestingly, the application of these preparations was essentially related to the adhesive properties thereof and the platelets only served to strengthen the architecture of the fibrin matrix.

A few years later the concept that these preparations could have healing and regenerative properties was developed. In the late 1980s, autologous “Platelet-Derived Wound Healing Factors” or PDWHFs, prepared through a two-step centrifugation process, were used in the treatment of chronic skin ulcers, which did not tend to heal. In 1997, a platelet concentrate referred to as a “platelet gel” began to be used in oral and maxillofacial surgery.

2. Platelet-Rich Plasma.

The term “platelet-rich plasma” (PRP) was first introduced to describe a platelet concentrate used for the treatment of severe thrombocytopenia. However, the use of the term PRP really began with Marx in 1998 when he published a comparative clinical study in which the regenerative potential of PRP was demonstrated in a series of patients undergoing mandibular reconstruction. PRP has subsequently been associated with the concept of platelet growth factors and the potential contribution thereof in inducing tissue healing.

According to the protocol for the production of PRP, the patient's blood is collected in test tubes containing anticoagulants and processed by means of two centrifugation steps. FIG. 2 diagrammatically shows the specific protocol. The PRP thus obtained can be applied to the site to be treated with a syringe or activated by thrombin and/or calcium chloride to trigger platelet activation and stimulate fibrin polymerization.

After the collection of blood in test tubes with anticoagulant, the first low-intensity centrifugation (soft spin) allows the separation of the blood into three distinct layers: red blood cells at the bottom, a cell plasma (platelet-poor plasma or PPP) at the top and a whitish layer referred to as the buffy coat, located therebetween, containing the highest concentration of platelets and leukocytes. For the production of pure PRP (P-PRP), the PPP and the buffy coat surface layer are transferred to another test tube and centrifuged at high intensity (hard spin), then most of the PPP and leukocytes are discarded and the P-PRP can be collected. To obtain the leukocyte rich PRP (L-PRP), the PPP, the entire buffy coat layer and some residual red blood cells are collected and transferred to another test tube to be centrifuged at high intensity (hard spin), then most of the PPP is discarded thus obtaining an L-PRP containing the buffy coat with most of the platelets and leukocytes, some residual red blood cells and PPP.

There are currently more than 20 different commercial systems for preparing PRP, which can lead to products with different features, in particular regarding the composition and the cellular concentration rate with respect to the baseline. On average, a 5-8× concentration is obtained, although a ratio of up to 11× has been reported with PRP.

3. Platelet-Rich Fibrin.

In 2001, a protocol was developed for producing a blood component referred to as the platelet-rich fibrin (PRF). In this case, blood is collected in test tubes without anticoagulant and centrifuged at a moderate speed. Three layers are thus formed inside the test tube: red blood cells and acellular plasma are located, respectively, in the lower and upper part thereof, while the fibrin clot, positioned therebetween, forms the PRF (FIG. 3). Since the PRF clot naturally forms inside the test tube, it has a strong fibrin matrix in which most of the platelets and leukocytes are trapped. Since the introduction thereof, PRF has undergone several developments: advanced PRF (a-PRF) was launched a few years ago and is characterized by reduced centrifugation speed and time, which allows for more uniform cell distribution inside the clot. Injectable PRF (i-PRF) has also been recently developed, which can be obtained with a further, even more delicate, centrifugation step. It has a liquid form, is very rich in white blood cells and can be used for infiltration into tissues and joints.

4. Plasma Rich in Growth Factors.

In parallel with the introduction of PRP and PRF, another platelet concentrate protocol referred to as the plasma rich in growth factors (PRGF) was suggested in 1999.

In short, blood is collected in test tubes with anticoagulant. After a low-intensity centrifugation, the red blood cells and the buffy coat layer are deposited on the bottom of the test tube and the plasma component on top of these. The latter is then manually separated into two fractions. The lower portion of about 2 mL, above the buffy coat, is PRGF, while the upper portion is plasma poor in growth factors (PPGF) (FIG. 4). PRGF can be applied as a liquid fraction in the target site or it can be pre-activated by adding 0.2 mL of 10% CaCl2) to induce clot formation.

5. Further Classifications of PRP.

PRP is further classified into four categories: activated, non-activated, leukocyte-rich, and leukocyte-poor. Activated PRP is prepared with calcium chloride with or without thrombin, which leads to the release of cytokines from platelet granules. Non-activated PRP preparations involve platelet contact with intrinsic collagen and thromboplastin, which activate platelets inside the connective tissue. Furthermore, the presence of leukocytes plays a role in inhibiting bacterial growth by improving soft tissue healing, which would have been hampered by infection.

In 2016, a DEPA classification was suggested based on injected platelet dose, production efficiency, purity, and activation of the PRP (FIG. 5).

Effectiveness, Fields of Application, and Action Mechanisms

Although the form thereof may vary based on different applications (liquid form, gel, membranes, or fibrin clots, etc.), as well as the preparation protocols, there is multiple clinical evidence on the efficacy of autologous platelet concentrates (APCs) in many fields of medicine, the fundamental role thereof in the regeneration of different tissues having been demonstrated: bones, cartilage, tendons, ligaments, muscles, skin, oral and vaginal mucosa, cornea, nerves, etc. However, the fields where APCs have been most studied and used are essentially oral and maxillofacial surgery, orthopedics and sports medicine, dermatology and aesthetic medicine, plastic and reconstructive surgery and ophthalmology.

The action mechanisms of PRP have not been fully elucidated, but laboratory studies have shown that the high concentration of growth factors can potentially accelerate the healing process. Growth factors promote wound healing by initiating the following steps: resolution of tissue necrosis, chemotaxis, cell regeneration, cell proliferation and migration, extracellular matrix synthesis, remodeling, angiogenesis, and epithelialization.

It has recently been discovered that the fibrin structure present on platelets supports the regenerative matrix, leading to the rapid organization of the correct morphological and molecular configuration for wound healing.

1. Clinical Evidence of the Efficacy of Autologous Platelet Concentrates (APCs) in Oral and Maxillofacial Regenerative Surgical Procedures.

In recent years, there has been a growing interest in the use of autologous platelet concentrates during oral regenerative surgical procedures as an additional tool to improve hard and soft tissue healing.

1.1. Autologous Platelet Concentrates (APCs) in the Healing of Post-Extraction Alveoli.

Several recent systematic reviews have evaluated the effectiveness of autologous platelet concentrates in improving alveolar recess healing after tooth extraction. Beneficial effects have generally been reported in terms of improved soft tissue healing, improved clinical and histological epithelialization of the wound margins and faster wound closure.

1.2. Autologous Platelet Concentrates (APCs) in the Treatment of Periodontal Defects.

Several systematic reviews have evaluated the efficacy of autologous platelet concentrates in the treatment of periodontal defects, including intraosseous defects, gingival recessions, and furcation defects. Beneficial effects on clinical and radiographic outcomes have been reported in the treatment of intraosseous defects. Conversely, PRP or PRF have not been shown to induce any clinical improvement in the treatment of gingival recessions or furcation defects.

1.3. Autologous Platelet Concentrates (APCs) in Endodontics and Endodontic Surgery.

Platelet concentrates have recently been used in the clinical treatment of immature necrotic teeth with the aim of regenerating the intracanal pulp and stimulating dental development, as well as in the surgical treatment of teeth with apical periodontitis to induce periapical tissue healing. There is clinical evidence on the benefits of using platelet concentrates in these conditions, but it is still poor.

1.4. Autologous Platelet Concentrates (APCs) in Maxillary Sinus Lift.

The use of platelet concentrates in combination with graft material during the maxillary bone augmentation procedure has been evaluated in both preclinical and clinical studies, with conflicting results.

1.5. Autologous Platelet Concentrates (APCs) in Dental Implantology.

Based on the evidence that platelet concentrates can promote bone regeneration, several animal studies have been conducted to evaluate the effect of PRP on the osseointegration process.

Histomorphometric analyses of the bone-implant interface in the early healing phase after implantation (6 or 8 weeks) revealed a significantly higher percentage of bone-to-implant contact in implants coated with liquid PRP than in those not bioactivated by PRP.

2. Clinical Evidence of the Efficacy of Autologous Platelet Concentrates (APCs) in Orthopedics and Sports Medicine.

The use of autologous platelet concentrates in the treatment of musculoskeletal conditions has become quite widespread in recent years. More recent literature has shown that PRP injections are relatively safe and can potentially accelerate or enhance the tissue healing process.

2.1. PRP in the Treatment of Tendon Injuries.

PRP has been most evaluated in the treatment of tendon injuries or tendinopathies. Tendons and ligaments heal through a dynamic process, with phases of inflammation, cell proliferation and subsequent tissue remodeling. Many of the cytokines present in PRP are involved in the signal transport, which occurs during this repair process. PRP also promotes neovascularization, which can not only increase the supply of blood and nutrients needed by cells to regenerate damaged tissue, but can also bring in new cells to remove tissue debris. Both of these action mechanisms are particularly interesting in chronic tendinopathies, as the biological environment can be unfavorable for tissue healing.

Important studies and several reviews demonstrate the short- and long-term efficacy of L-PRP or PRP in the treatment of lateral epicondylitis, chronic refractory patellar tendinopathy, rotator cuff tendinopathy, and in the management of chronic plantar fasciitis. In contrast, studies supporting the use of PRP in Achilles tendinopathy have provided conflicting results.

2.2. PRP in the Treatment of Osteoarthritis.

The idea of using PRP for cartilage regeneration is based on in vitro results reported in scientific literature, which show that the growth factors released by alpha platelet granules can increase the synthetic capacity of chondrocytes through the up-regulation of gene expression, the production of proteoglycans and the deposition of type II collagen.

Clinical trials on the use of PRP in cartilage lesions have involved patients with knee or hip osteoarthritis.

2.2.1. PRP in the Treatment of Knee Osteoarthritis.

Several studies have evaluated the effectiveness of intra-articular PRP injections in the treatment of knee osteoarthritis. PRP was compared with placebo, other alternative injections (corticosteroids, hyaluronic acid), oral medications, ozone therapy, and lifestyle changes.

14 randomized studies comprising 1,423 patients were reviewed in a meta-analysis. The individual studies considered several preparations of autologous platelet concentrates (APCs) including PRP, L-PRP, and plasma rich in growth factors (PRGF). The meta-analysis demonstrated that multiple injections of APCs showed a significant improvement (score calculated with WOMAC osteoarthritis index) in the follow-up at 3, 6 and 12 months with respect to the controls, furthermore the use of non-APCs showed an increased risk of adverse post-injection effects. By virtue of this meta-analysis it was possible to conclude that intra-articular injections of APCs are more effective in the treatment of knee osteoarthritis with respect to other alternative injections.

2.2.2. PRP in the Treatment of Hip Osteoarthritis.

Compared to knee osteoarthritis, studies on the effects of PRP in the treatment of hip osteoarthritis are rather limited. Among these at least four randomized controlled trials refer to the comparison between PRP injections for hip osteoarthritis and hyaluronic acid (HA) injections. These studies demonstrated that treatment with PRP initially shows a better reduction in pain than treatment with hyaluronic acid, however this initial advantage seems to decrease over time and at 12 months the two treatments, with PRP and with HA, show very similar efficacy with each other. Overall, the intra-articular injection of PRP for hip osteoarthritis has been shown to be safe and effective in reducing pain and restoring function.

2.3. PRP in the Treatment of Degenerative Disease of the Intervertebral Discs.

PRP treatment for intervertebral disc repair has been the subject of intense research with promising results. For example, a study was conducted with the aim of evaluating the effect of a single intradiscal injection of PRP in the degenerated intervertebral disc of rabbit and to observe the regeneration process over a follow-up period of 6 weeks.

The discs L3-L4 and L4-L5 of 18 adult female rabbits were injured according to a well-defined degenerative model and the animals were divided into two groups: after the injury, the group A rabbits received an intradiscal injection of autologous PRP in the discs, while in the control group (group B) the same procedure was followed by intradiscal injection of normal saline in the discs. During the 6-week follow-up, a noticeable progression of the degeneration process was observed in group B, while the degree of degeneration was significantly lower in group A, where the regeneration of the intervertebral disc and the reversal of the injuries were instead apparent. The study concluded that intradiscal treatment with PRP in degenerative intervertebral disc disease results in the maintenance of the basic morphological features of the disc, with apparent regeneration after injury.

2.4. Autologous Platelet Concentrates (APCs) in the Enhancement of the Post-Surgery Healing Process.

Autologous platelet concentrates have been successfully used to promote post-surgery repair of tendons and ligaments.

2.4.1. Autologous Platelet Concentrates (APCs) in Rotator Cuff Repair.

Several major clinical studies have evaluated the effects of APCs in enhancing arthroscopic repair of rotator cuff tears. Many studies have specifically analyzed the use of platelet-rich fibrin (PRF), while other studies have analyzed the use of PRP injected directly into the repair site. Evidence from randomized clinical trials does not demonstrate an absolute benefit of PRP in enhancing the healing process in rotator cuff repair surgery, but does demonstrate that activated PRF, administered at the bone-tendon interface in combination with the suture bridge repair technique, is capable of giving good results. Some additional, albeit limited, data have shown that PRP may be useful in reducing postoperative pain and repairing small and medium lacerations.

2.4.2. PRP in Achilles Tendon Repair.

The effects of PRP in enhancing healing during Achilles tendon rupture have been quite promising in preclinical models. Indeed, most rodent studies show a beneficial effect of platelet concentrates on the healing of acute Achilles tendon ruptures when used as adjunct therapy. On the other hand, clinical trials on humans, with respect to the use of PRP in the repair of acute Achilles tendon tears, are rather limited and the results thereof are somewhat conflicting.

2.4.3. PRP in Anterior Cruciate Ligament Surgery.

Studies on the use of PRP in anterior cruciate ligament reconstruction surgery have focused on 3 biological processes: (1) osteo-ligamentous integration of the graft inserted in the two intraosseous tunnels made in the femoral condyle and in the tibial plateau, (2) maturation of the joint part of the graft (the neo-ligament assumes the morphology of the native ligament), (3) healing of the autologous graft site and pain reduction.

Early studies have shown no significant clinical effect of PRP on graft integration or maturation, but more recent studies have shown promising results in reducing pain at the autologous ligament removal site.

2.5. Autologous Platelet Concentrates (APCs) in the Treatment of Muscle Injuries.

In vitro studies have demonstrated that PRP is capable of leading to the proliferation, but not to the differentiation of myoblasts, while treatment with PPP induces the myoblasts to differentiate into muscle tissue. This suggests that perhaps the most beneficial treatment of muscle injuries may be with PPP, although in vivo studies will be needed in animals, followed by human clinical trials, to further investigate this treatment option.

2.6. PRP in the Management of Fractures and Nonunions.

Most preclinical studies support the use of PRP to improve bone healing. This is mainly due to the acceleration and increased induction of bone regeneration demonstrated in fracture models treated with PRP. Furthermore, PRP treatment has been demonstrated to improve bone strength in a rodent osteotomy model.

Despite the positive results of preclinical studies, there is no unanimous consensus, based on important clinical studies, to support the routine use of PRP to improve bone healing.

As regards the treatments of nonunions, only one randomized clinical study was identified which reported a comparison of clinical outcomes. This study failed to show a benefit of PRP over bone morphogenetic protein 7 or BMP-7 (which is the gold standard therapy) in the treatment of tibial nonunions.

3. Clinical Evidence of the Efficacy of Autologous Platelet Concentrates (APCs) in Dermatology and Aesthetic Medicine.

PRP has been successfully used in dermatology for skin rejuvenation and for the treatment of various diseases such as vitiligo, acne, alopecia and stretch marks. Furthermore, favorable results have been recorded in aesthetic gynecology.

3.1. PRP in the Treatment of Vitiligo.

Vitiligo, an acquired pigment disorder of unknown origin, is the most frequent cause of depigmentation worldwide, with an estimated prevalence of 1-2%. Vitiligo is clinically characterized by the development of white macules due to the loss of functioning melanocytes in the skin, hair, or both. The disorder can be psychologically devastating, especially in dark-skinned individuals.

Vitiligo excimer laser therapy, one of the most widely used therapies today, generally takes months or years to achieve excellent results. Therefore, a study was conducted to evaluate the additive effect possibly obtained with the use of PRP, with respect to the use of the excimer laser alone, in the therapy of vitiligo. For this comparative study 52 patients with stable (no new lesions for 6 months), non-segmental and symmetric vitiligo, were divided into two groups. Patients in group I were treated with intradermal injections of PRP and excimer laser, while patients in group II were treated with excimer laser only. The PRP injections were repeated every 3 weeks for 4 months and the excimer laser twice a week for 16 weeks until complete response. During the 3-month follow-up, patient satisfaction was assessed, the appearance of any complications was monitored and a clinical (re-pigmentation response) and histopathological evaluation of the lesions was performed. In group I, compared to group II, a higher and statistically significant response to treatment was observed. Furthermore, there was a significant difference in patient satisfaction between the two groups and no significant side effects were reported. The study authors concluded that the combination of PRP and excimer laser phototherapy represents an effective treatment for vitiligo, as PRP increases the effectiveness of the laser therapy, improving the outcome.

3.2. PRP in the Treatment of Acne.

Acne is a multifactorial disease associated with the excessive proliferation of Propionibacterium acnes, characterized by a chronic disease of the pilosebaceous follicle. Clinical signs include papules, pustules, comedones, cysts, abscesses and lesions, which can sometimes leave atrophic scars which are the most serious complication of acne. The most commonly used topical drugs are retinoids and antimicrobials, which are effective in cases of mild acne. However, such treatments tend to have a limited therapeutic effect and multiple adverse effects such as erythema, peeling, dry skin, microbial resistance, and photosensitivity. When acne does not respond to topical treatments (cases of moderate to severe acne), systemic therapy is used, which consists of the administration of oral antibiotics. This therapeutic option results in unwanted side effects for the patient, such as photosensitivity, gastrointestinal disturbances, liver dysfunction (in severe cases), hypersensitivity reactions and increased bacterial resistance. These reasons support the need to develop minimally invasive therapeutic strategies, without risk of toxicity. The use of PRP as a single therapy or with another concomitant treatment (microneedling, oral or topical antibiotics and laser) has been suggested with satisfactory results. In vitro studies have shown that PRP can inhibit the growth of Propionobacterium acnes. Furthermore, the inflammation caused at the moment of PRP inoculation triggers a cell stimulus which contributes to the production of collagen which determines an improvement in the skin's appearance.

Some studies have been conducted with favorable results using PRP as the only acne treatment or in combination with other treatment options. In one of these studies, three doses of PRP were applied over a period of 3 months, one every month, to a patient who had been suffering from acne for 3 years, observing a significant reduction in both inflammatory lesions and scarring generated by the acne.

In another study, PRP with microneedling was used to treat a group of patients with atrophic scars secondary to acne, while a control group was treated with distilled water and microneedling, finding that PRP combined with the microneedling technique is more effective in the treatment of atrophic acne scars with respect to microneedling alone.

In another of these studies it was observed that the use of intradermal PRP potentiates the effects of CO2 laser therapy in the treatment of secondary acne scars.

3.3. PRP in the Treatment of Alopecia.

Alopecia is defined as the progressive loss of hair. There are two types of alopecia: cicatricial and non-cicatricial alopecia. The latter type of alopecia, within which androgenetic alopecia and alopecia areata are included, has a better prognosis because the hair follicles have not been completely lost. Androgenetic alopecia affects both men and women and is the most common. There are currently only two treatments approved by the Food and Drug Administration for alopecia: topical minoxidil and oral finasteride. These treatments have several adverse effects, such as headache and hypertrichosis associated with minoxidil and reduced libido associated with finasteride, a treatment, which has been tested only in men. New treatment strategies are currently being developed, searching for good tolerance and greater efficacy. For example, PRP has been used in several studies for the treatment of non-scarring alopecia, obtaining positive results, such as the growth of new hair, resistance at the root and the increase in hair thickness. In one of these studies, patients with androgenetic alopecia were treated with three PRP injections, one every 20 days, and a final reinforcement injection at 6 months, finding a decrease in hair loss and an increase in hair density from the first 3 months, without showing adverse effects.

The efficacy of PRP was also tested in addressing alopecia areata: 20 patients with alopecia areata who had not responded to previous treatments were treated in a study, obtaining significant improvements based on clinical follow-up evaluations performed one year later.

The action mechanism of PRP in countering alopecia is not very clear, although it seems to be linked to the action of the growth factors. Fibroblastic growth factor stimulates papilla cell proliferation, leading to hair elongation. PDGF, EGF and VEGF seem to act in activating the proliferative phase of the hair, originating a new follicular unit.

3.4. PRP in the Treatment of Stretch Marks (Striae Distensae).

Striae distensae (SD) are cutaneous scars associated with atrophy of the epidermis. The areas most frequently affected are the outer surface of the thighs and lower back in boys, the thighs, upper arms, buttocks, and breasts in girls. DS affects up to 90% of pregnant women, 70% of girls and 40% of boys during puberty.

The pathogenesis of DS remains ambiguous. Several theories have been suggested such as mechanical lengthening caused by weight gain, corticosteroid therapy, and hormonal changes resulting from pregnancy and during puberty. The results of SD treatments are still disappointing.

PRP has recently been suggested as a new treatment for DS. A study evaluated the effect and safety of intralesional injection of PRP, compared to topical 0.05% tretinoin, in the treatment of DS. 30 patients (27 females and 3 males) with bilateral striae distensae were enrolled in this study. In each patient the SDs of one side of the body were treated with intralesional injections of PRP, those of the other side with topical tretinoin. Skin biopsies were taken from both sides before and after treatment. Clinical improvement was evaluated blind by 2 dermatologists, in addition to patient satisfaction. The study authors showed a statistically significant improvement in both treatments, but the improvement was greater in the SDs treated with PRP injections. Patient satisfaction also showed that improvement was more significant on the PRP side. In the dermis, collagen and elastic fibers increased in all the post-treatment biopsies. The conclusion of this study was that PRP injection and topical tretinoin are safe for the treatment of DS, but PRP is more effective and gives a better therapeutic response than tretinoin.

3.5. Use of PRP in Skin Rejuvenation.

Aging is commonly defined as a progressive loss of the skin's homeostatic capacity. It is a complex process which occurs as a result of extrinsic factors, such as UV radiation, environmental pollutants, and exposure to chemicals, among others. One of the most important factors is solar radiation, this generates free radicals which increase the activity of collagenase by activating the degradation of collagen and decreasing the concentration of transforming growth factor (TGF), as well as reducing the formation of new collagen fibers. Among the intrinsic factors, hormone levels, genetic regulation and inflammatory factors generate molecular changes at both the cellular and histological level. At the anatomical level, the main changes are represented by wrinkles, benign neoplasms, a decrease in the amount of basal keratinocytes and, over time, a decrease in the aqueous content of the tissues which alters the hydration status of the skin. All these changes not only affect physical appearance, but also have physiological implications as the skin, in a state of aging, does not adequately perform the protective barrier function thereof. The main treatment to combat skin photoaging is to prevent sun exposure. However, there are other secondary prevention options, such as retinoid preparations, antioxidants, oral estrogens, and even several tertiary prevention options, such as chemical peels, lasers, botulinum toxin, skin and subdermal fillers. However, none of these alternatives are a natural, autologous, and chemical-free strategy.

Biostimulation is a restorative treatment, which consists of restoring the metabolism and proper functioning of the skin, based on the use of PRP to biologically activate the anabolic functions of the fibroblast and the production of collagen III and IV, elastin, and hyaluronic acid. Thanks to the growth factors thereof, PRP acts on target cells and on the extracellular matrix, thus obtaining tissue repair and regeneration. Several studies have shown that PRP produces noticeable changes on aged skin, restoring vitality, increasing skin levels of collagen, recovering elastic texture, improving vascular flow and stimulating softness, tone, and appearance.

PRP has also been used in combination with other typical anti-aging treatments with satisfactory results. In one of these studies, an average of three sessions of PRP plus hyaluronic acid were used in 94 women with varying degrees of facial aging. It was observed that an improvement in fine wrinkles and skin tone was apparent already from the second application; by the end of the sessions most of the wrinkles had disappeared with a clear rejuvenation of the face.

In another study it was shown that the combination of PRP and ultrapulsed fractional CO2 laser reduces the duration of laser side effects and improves treatment efficacy after 3 months.

3.6. PRP in Aesthetic Gynecology.

Platelets have been shown to release around 35 factors which promote tissue regrowth, healing, and regeneration. These abilities have been used by aesthetic gynecologists in treatments such as vaginal rejuvenation.

3.6.1. PRP in Vaginal Rejuvenation.

Cosmetic professionals have used PRP for the regeneration of the mucous membrane and muscle layer of the vagina. After the injection of PRP the vaginal vascularity increases, with a consequent notable increase in sensitivity, while the skin of the external genitalia becomes thicker and firmer. Furthermore, the ligaments and muscles which support the urethra become stronger, relieving the problem of urinary incontinence. In a study on the use of PRP in a case of vaginal rejuvenation it was shown that the graft of autologous adipose tissue (lipofilling) mixed with PRP, in a patient with vaginal atrophy, had solved the typical symptoms of atrophy and restored the shape and volume of the vagina. Furthermore, it was highlighted that the rejuvenated appearance of the external genitalia had provided the patient with a pleasant aesthetic result.

4. Clinical Evidence of the Efficacy of Autologous Platelet Concentrates (APCs) in Plastic and Reconstructive Surgery.

By virtue of the ability thereof to stimulate and accelerate soft tissue healing, there is a great deal of evidence on the efficacy of APCs in the treatment of burns, wounds and especially skin ulcers.

4.1. PRP in the Treatment of Burns.

Standard burn treatment includes two main steps: the first is the removal of necrotic tissue, which must be carried out within the first 48 hours, which reduces the risk of bleeding and infections, as well as the associated morbidity and mortality. The second is skin grafting, used to accelerate healing and minimize scarring. Despite these measures, complications and obstacles still exist in the treatment of burns, as both secondary infections and long healing periods continue to pose a challenge. In this sense, PRP applied in the treatment of burns could favor the acceleration of cell repair.

In an experimental study, burns were induced in 10 rats and PRP was subsequently applied thereto. After 7 days the rats were sacrificed to study the histological changes and determine the amount of hydroxyproline (amino acid component of collagen which is important in tissue regeneration processes). The PRP-treated rats showed higher hydroxyproline levels and less inflammatory cell infiltration than the control group, which had not undergone any treatment. However, there were no significant histological differences (development of fibroblasts, blood vessels and degree of epithelialization).

In another study, PRP was used locally in a group of rats suffering from deep epidermal wounds from second-degree burns associated with diabetes mellitus and in a second group of rats with third-degree burns. The authors of this other study showed that PRP accelerated the healing process by promoting the formation of granulation tissue and new epidermis in both groups, although it was less effective in the treatment of the third-degree burns.

Furthermore, it has been described that the subcutaneous injection of PRP in rats can mitigate burn-induced neuropathic pain.

With regard to human clinical studies, the effect of PRP on wound healing was evaluated in 52 patients with burns on several body areas and for whom skin grafting surgery was required, observing how a better epithelialization rate was obtained with PRP.

4.2. Autologous Platelet Concentrates (APCs) in the Treatment of Wounds (Traumatic and Surgical).

The use of APCs has also been evaluated for wound healing, albeit to a lesser extent than in other dermatological applications. “Wound” is intended as a continuity defect of the skin and soft tissues resulting from local trauma or surgery.

Wound healing represents the body's ability to repair injured tissue. However, there are several obstacles to tissue repair, which can be classified into general or systemic factors, directly mediated by the patient's general state of health, and local factors. As a result of the effect of these factors, wound healing disorders can occur such as: seromas, hematomas, soft tissue necrosis, dehiscence, hypertrophic scar formation, keloids.

A controlled study was recently conducted on 59 patients with acute traumatic wounds, which did not require skin flap grafts. With respect to Vaseline gauze, the weekly topical application of autologous PRP gel for 3 weeks produced early improvements in the wound area, highlighted during follow-up starting at week 1, as well as lower pain scores at week 2 and week 3.

Skin flap grafting is one of the most common tissue transplants for wound repair. In an experimental study, the efficacy of PRP gel in increasing the survival rate of the transplanted skin flap using Sprague-Dawley (SD) rats was evaluated. The study found that PRP gel increases the survival rate of the skin flap, reduces the inflammatory response, and has better effects in terms of generating new soft tissues. Based on the results obtained, it was concluded that the efficacy of PRP gel in skin flap transplantation is satisfactory and that PRP could be a new clinical method to promote skin flap survival.

4.3. PRP in the Treatment of Skin Ulcers.

An ulcer is a lesion, which can arise spontaneously or due to an underlying trauma or disease which generates a skin defect. Ulcers usually affect the lower limbs and are considered chronic when they persist for more than 6 weeks, even after receiving adequate treatment.

These lesions, regardless of the origin thereof (venous, arterial, diabetic and pressure), are of great importance because in most cases they can compromise mobility, resulting in loss of work productivity and high health care costs. The incidence of these lesions is thought to be related to aging, although factors such as atherosclerotic occlusion, obesity and diabetes increase the risk of developing an ulcer.

The basic principles of skin ulcer management are to treat the underlying cause as well as to allow adequate function of the affected area. Current treatments include surgery, sclerotherapy, conventional therapy (debridement, bandages, moisturizing dressings, topical antibiotics) and adjuvant drug therapy. It is the aim of the treatment for these lesions to close the wound as quickly as possible, in order to avoid possible complications and prevent it from becoming chronic. However, despite treatment, various ulcers do not heal properly, persist for months and even years, and may recur even after healing. These lesions irreparably deteriorate the affected area, thus increasing the chances of amputations.

PRP represents an advance in the search for new therapeutic options, by virtue of the ability thereof to stimulate and accelerate soft tissue healing, creating an environment, which is more conducive to the restoration of the affected areas. The use of PRP for the treatment of various types of skin ulcers has been described with satisfactory results. For example, in one of the first studies related to the treatment of chronic skin ulcers with PRP, it was reported that after 8 weeks of treatment there was a reduction of the lesion surface of about 73%. In a clinical case, after more than 4 months of unsuccessful treatments, an elderly woman's pressure ulcer was treated with PRP and the total closure thereof was achieved in 57 days. Another clinical case reports that in 12 patients, with an average age of 33.5 years, 17 varicose ulcers were treated, resulting in a reduction of more than 94% in the area affected by the ulcers. A team of researchers used topical PRP (gel) administrations on 24 patients, with a 90% reduction in the ulcer area in 70.8% of patients. Another study compared the efficacy of PRP versus conventional topical antibiotic treatment in 56 patients with diabetic foot ulcers, achieving a cure rate of 86% in the PRP group and only 68% in the control group.

Although these studies suggest that PRP may become a safe, inexpensive, and effective therapeutic alternative for the treatment of chronic non-healing ulcers, there is still no clear evidence on the efficacy of PRP in healing lesions with obvious complications.

4.4. PRP in the Treatment of Keloids and Hypertrophic Scars.

Keloids and hypertrophic scars are two types of exuberant pathological scars. With respect to hypertrophic scars, keloids are characterized by more serious clinical aspects, as they are more frequently the cause of itching and pain. Keloids also progress to form thick, solid scars, which rarely heal spontaneously. There are several prevention and treatment options for keloid management, but recently some researchers have focused on the role of PRP in modifying keloid pathology. In vivo studies have demonstrated that PRP increases dermal fibroblast proliferation, collagen expression and matrix protein synthesis. Injected into the wound bed, PRP is currently being studied as a post-surgery therapy in keloid excision procedures. One clinical study reported 29% keloid recurrence at two years when PRP is used intra-operatively, during surgical excision, and post-operatively, on a monthly dosing regimen for three months. This study suggests the potential of PRP in modifying the abnormal wound healing typically seen after the surgical excision of keloids.

4.5. PRP in Breast Reconstruction.

Together with autologous adipose tissue, PRP has been used successfully in breast reconstruction. One study enrolled 100 patients between the ages of 19 and 60 with soft breast tissue defects. The patients were divided into two groups of equal size. The experimental group was treated with PRP and autologous fat grafts, while the control group was treated only with autologous fat grafts (autologous lipofilling). This study highlighted that the patients treated with PRP added to autologous fat grafts showed a 69% retention rate of the shape and three-dimensional volume of the breast one year after the intervention, while the control group patients showed a 39% retention rate. The study authors concluded that PRP treatment mixed with autologous fat grafts resulted in significant improvements in the retention of breast shape and volume in patients with soft breast tissue defects. Similar results were obtained in another similar study.

5. Clinical Evidence of the Efficacy of Autologous Platelet Concentrates (APCs) in Ophthalmology.

Platelet-rich plasma in ophthalmic formulation (E-PRP) has recently been used with positive results in the treatment of various eye diseases. The preparation of E-PRP in the two available formulations, eye drops and clot, is low-cost and easy, even if it requires compliance with strict sterility conditions. No serious adverse effects have been described with the use of these products and the treatment is generally well tolerated.

5.1. Autologous Platelet Concentrates (APCs) in the Treatment of Persistent Corneal Epithelial Defects.

A persistent corneal epithelial defect (PED) is defined as a lesion, which measures more than 2 mm in diameter, persists for more than 2 weeks and is resistant to conventional treatments. Lacrimal and neurogenic dysfunctions are the two main causes of PED, although the etiopathology thereof is quite variable. Other conditions which can develop in PED include burns, immunological factors, infections, epithelial dystrophies, metabolic changes, and trauma. Conventional treatments such as artificial tears, therapeutic contact lenses, tarsorrhaphy, anti-inflammatory agents and oral antibiotics usually do not improve PED symptoms; consequently a resistant PED can continue to degenerate, leading to progressive stromal lysis and subsequent perforation.

Researchers have published a work on a total of 34 eyes with persistent corneal epithelial defects post-keratoplasty (24 penetrating keratoplasty and 10 deep anterior lamellar keratoplasty), refractory to conventional medical treatments, treated with PRP eye drops every 3 hours. The treatment with autologous PRP eye drops resulted in rapid re-epithelialization in all the eyes. The average duration of treatment for complete re-epithelialization was significantly shorter than the average duration of treatment for conventionally treated corneal defects. The researchers concluded that treatment with autologous PRP eye drops is an effective and reliable approach, which accelerates the re-epithelialization of persistent post-transplant corneal epithelial defects.

Other investigators have compared the efficacy of PRP-induced corneal epithelial healing with that induced by autologous serum (AS) in patients with a persistent corneal epithelial defect resulting from a post-infectious inflammatory process. AS was used in 17 eyes and PRP in 11 eyes of 28 patients. The healing rates of the corneal epithelia of the eyes treated with PRP were significantly higher than those treated with AS. The researchers concluded that PRP was shown to be effective in the treatment of persistent corneal epithelial defects after infectious keratitis.

The effect of PRGF on the PEDs was evaluated in two prospective studies involving 26 and 18 eyes, respectively. Both studies showed an average total recovery from the epithelial defect in 89% of cases. Ocular inflammation and pain were the other two parameters, which had clearly improved in most cases after treatment with PRGF. Visual acuity corrected with glasses also improved in 58% of cases. In both studies, the application of PRGF proved highly effective in the treatment of PEDs, even in those cases which had previously been treated with AS, as in the case of one third of the patients analyzed.

In another study involving 35 patients, the efficacy of PRGF in the treatment of type II and type III corneal burns (Dua classification) caused by alkaline substances was evaluated. The results obtained showed a significant reduction in corneal epithelialization time in patients with type II and III burns treated with PRGF with respect to the control groups (who had received conventional topical treatment) and with respect to the group with type III burns subjected to treatment with autologous blood (autohemotherapy). The corneal and conjunctival healing times were also reduced in the PRGF groups with respect to the control and autohemotherapy groups. No differences were found between patients treated with autohemotherapy and those treated with traditional medical treatments. These results suggest that applying PRGF to ocular alkaline burns is effective and carries no risk of unwanted side effects.

5.2. Autologous platelet concentrates (APCs) in the treatment of dry eye syndrome.

Dry eye syndrome (DES), also known as keratoconjunctivitis sicca (KCS), is a disorder of the tear film caused by an alteration in the amount of tears and/or the composition thereof; it is considered an inflammatory process which affects both the lacrimal gland and the ocular surface.

Two studies conducted in recent years to evaluate the efficacy and safety of PRGF-based eye drops in the treatment of DES are reported in a review. A total of 34 patients with moderate to severe DES were included in these studies. PRGF was applied topically as eye drops 4-6 times a day for 1-3 months. The results obtained in these studies after treatment with PRGF showed that 82% of patients showed a substantial improvement or complete disappearance of symptoms related to dry eye syndrome. No cases of poor tolerance or undesirable effects attributable to the use of PRGF were reported in any of the studies. These results suggest that PRGF-based eye drops have great potential in reducing signs and symptoms of moderate to severe DES.

In another study, the results of hyposecretory dry eye treatment with PRP or sodium hyaluronate (SH) artificial tears, respectively, were evaluated and compared. To carry out this study, 83 patients were divided into two groups based on the treatment applied. In both eyes of the PRP group, compared to the SH group, at 15 and 30 days of treatment there was a significantly greater reduction in symptoms, visual improvement, a reduction in hyperemia and in corneal and conjunctival staining with fluorescein, an increase in the Schirmer test result and a reduction in tear osmolarity. The authors of this study concluded that the treatment of hyposecretory dry eye with PRP induces a more significant positive effect on symptoms with respect to treatment with SH, especially in moderate and severe cases.

Other researchers conducted a study to evaluate the effectiveness of platelet-rich plasma (PRP) injections in the treatment of severe dry eye. 30 patients with severe dry eye who had been diagnosed with Sjogren's syndrome were enrolled in the study. The patients were divided into two groups: the experimental group received PRP injections on days 0, 30, 60, and 90, as well as hyaluronic acid five times a day. The comparison group received hyaluronic acid five times a day. The subjects were monitored at baseline and at 30, 60, and 90 days. The PRP (1 mL) was injected transcutaneously into the anatomical area corresponding to the position of the lacrimal gland. The primary outcomes were changes in corneal staining according to the Oxford classification, Schirmer's test results, and tear film break-up time (TF-BUT). The secondary outcomes were changes in dry eye-related symptoms on the Ocular Surface Disease Index (OSDI) questionnaire and treatment adherence. All the subjects completed the study. The experimental group showed improvements in all primary outcome measures with respect to the control group. An improvement in subjective values was also found based on the OSDI questionnaire. The researchers concluded that the injection of PRP is safe and effective in improving tear and subjective parameters and has been shown to be superior to hyaluronic acid in the management of patients with severe dry eye.

5.3. Autologous Platelet Concentrates (APCs) in the Treatment of Post Laser-Assisted In Situ Ocular Surface Keratomileusis Syndrome.

Laser-ASsisted In situ Keratomileusis (LASIK) is currently the most widely used procedure in the world to correct refractive errors such as myopia, hyperopia, and astigmatism.

Post-LASIK ocular surface syndrome (OSS) is a term used to describe a wide range of symptoms such as dry eye, persistent neurotrophic epitheliopathy, tear film instability, tear deficiency, reduced acuity and visual quality, and neuropathic pain, which can affect 0.8% to 20% of patients who have undergone LASIK.

To evaluate the efficacy of autologous platelet-rich plasma (E-PRP) based eye drops in the treatment of chronic post-LASIK ocular surface syndrome, researchers conducted a clinical study including 156 eyes from 80 patients with this condition, who were treated with autologous E-PRP 6 times a day as monotherapy for 6 weeks. By the end of the sixth week of treatment, all the dry-eye symptoms had improved in 85% of cases. In 89.6% of patients, who had positive corneal fluorescein staining (CFS) before treatment, a reduction from at least one quadrant was observed until the CFS was completely clear. Of the 113 eyes which had chronic punctate keratitis at baseline, 101 (89.4%) showed significant improvement after treatment with E-PRP. Conjunctival hyperemia improved in 93.3% of patients with previous signs of ocular surface inflammation. There was also a significant improvement in acuity and visual quality. The researchers concluded that autologous E-PRP monotherapy is a well-tolerated, safe, and effective treatment for the management of post-LASIK ocular surface syndrome.

5.4. Autologous Platelet Concentrates (APCs) in Corneal Surgery.

Severe ocular surface disease (OSD) due to trauma, thermal and chemical burns, Stevens-Johnson syndrome (SJS), ocular cicatricial pemphigoid (OCP) or other conditions, currently represent a serious clinical challenge for ophthalmologists worldwide. In these cases the corneal epithelial stem cells, located in the corneal limbus, are destroyed and the spontaneous physiological healing of the ocular surface is no longer possible. This is followed by the formation of ulcers, corneal perforation, or coverage of the corneal surface by neighboring conjunctival epithelial cells with consequent neovascularization, chronic inflammation, neoformation of fibrous tissue and stromal scarring. The treatment of ocular surface disorders has a multifactorial approach, although conventional treatments are often insufficient for solving the problem. In patients with severe ocular surface disease, the success of amniotic membrane transplantation in the treatment of ulcers and/or perforations is less than 20%.

In a review, the use of solid ophthalmic PRP (E-PRP), in combination with amniotic membrane, bovine pericardium and autologous fibrin membrane, as an adjuvant in the surgery of severe corneal ulcers and perforations was analyzed. The review authors concluded that solid E-PRP, in the form for use in ophthalmology, is a reliable and effective surgical adjuvant to promote corneal wound healing in severe corneal ulcers and corneal perforations and may be associated with other ocular surface reconstruction procedures.

5.5. Autologous Platelet Concentrates (APCs) in the Treatment of Chronic Ocular Hypotonia after Glaucoma Surgery.

Hypotonia is generally defined as an intraocular pressure (IOP) of 5 mmHg or less. Following a filtering procedure for glaucoma, optical hypotonia can cause serious visual complications such as macular edema, hypotonic maculopathy, corneal edema, exudative retinal detachment, etc.

Researchers have described a novel approach for the treatment of severe ocular hypotonia secondary to glaucoma filtering surgery with mitomycin C, injecting autologous platelet-rich plasma (E-PRP) into the anterior chamber to block excessive filtration through an abnormally thinned sclera. A patient with chronic severe hypotonia and corneal edema following filtering surgery for glaucoma with mitomycin C received a single injection of 0.3 mL of autologous platelet-rich plasma into the anterior chamber. As early as 6 hours after the procedure, the intraocular pressure had improved and remained stable for the entire 6-month follow-up period. No abnormalities in filtration, hypotonia or other complications were observed. The researchers concluded that intracameral platelet-rich plasma (E-PRP) injection is a rapidly effective and safe procedure for the treatment of severe chronic ocular hypotonia following filtering surgery for glaucoma.

6. Clinical Evidence of the Efficacy of Autologous Platelet Concentrates (APCs) in Veterinary Medicine and Surgery.

All of the above can be entirely transferred to veterinary medicine and surgery, also considering the fact that most of the above experiments were conducted on animal models and subsequently the results obtained were used to perform similar studies on human patients.

As can be seen from the several studies reported above, for many applications of autologous platelet concentrates (APCs) the results obtained were not satisfactory, and even for those applications for which the effects of treatment with APCs have been positively judged, the results obtained are clearly susceptible to further notable improvements.

Therefore, an attempt was made to enhance the effect of APCs by using them in combination with adult stem cells taken from the same patient, antibiotics, anti-inflammatory agents, vitamins, etc., however the increase in efficacy obtained with these combinations, with respect to the use of APCs alone, was quite modest. Therefore, it was assumed that the limited efficacy of the various preparations was due to a low platelet concentration and, consequently, to a low concentration of the factors contained in the platelet granules.

In this regard it is important to consider that human autologous platelet concentrates (APCs) are considered blood components for non-transfusional use, the preparation and use of which is represented in European legislation by Directive 2005/61/EC of the Commission of 30 Sep. 2005 which applies Directive 2002/98/EC. This European reference legislation is better defined by each nation with its own national law.

Currently in Italy, the procedure for the preparation and use of APCs must be performed according to the provisions set out in Italian Ministerial Decree of 2 Nov. 2015, “Provisions related to the quality and safety requirements of blood and blood components”, which defines some parameters which must absolutely be respected, such as:
blood sampling volume not exceeding 60 mL per single procedure; in a cycle of procedures, the total volume withdrawn must not exceed 300 mL in 90 days;
for platelet concentrates, the platelet concentration must be 1×10⁶/μL±20% and the volume commensurate with the type of use.

The aforementioned Italian decree has been reported as a guide for Good Clinical Practices (GCP) and indeed in almost all published international studies a platelet concentration ranging between 1.0-1.5×10⁶/μL is used, in line with the provisions of Italian legislation. This platelet concentration (usually in a 5 mL volume of plasma) is scientifically supported by evidence that lower concentrations are not effective enough in promoting bone and soft tissue healing and regeneration. It has also been seen that higher concentrations are not applicable for legislative reasons, but even in cases where these higher concentrations have been used in experimental studies, they have substantially shown the same efficacy as the standard concentrations. The same can be said for closer cycles of procedures, using withdrawals exceeding 300 mL in 90 days.

There is therefore a need to have preparations based on natural substances, of low cost and without side effects, which do not alter the action of the autologous platelet concentrates, but which, on the contrary, amplify the effectiveness thereof.

### SUMMARY OF THE INVENTION

The inventors of the present patent application have surprisingly found that a formulation according to the appended claims is capable of solving the problems of the compositions known in the art. In fact, they have found that by combining the mixture of factors extracted from colostrum in active form with platelet concentrates (PCs), the activity of the latter is unexpectedly enhanced in all fields of application.

### OBJECT OF THE INVENTION

In a first object, the present invention relates to a composition comprising platelet concentrates (PCs) and a colostrum isolate mixture rich in biological factors.

In an aspect of the invention, the composition is topical or injectable.

In a preferred aspect, the composition comprising further compounds having specific properties.

According to an aspect of the invention, the formulations described are in the form of liquid solutions, lotions, foams, sprays, creams, salves, ointments, pastes, gels, membranes, clots, powders or other suitable forms of administration. The administration can occur topically, by injection (intraepidermal, intradermal, subcutaneous, transcutaneous, intramuscular, intraarticular, intraocular), vaginally, ocularly, transdermally or by other suitable routes of administration.

In a second object, the present invention relates to the medical use of the described composition.

The multiple medical uses represent further aspects of the present invention.

### BRIEF DESCRIPTION OF THE DRAWINGS

FIG. 1 shows the factors contained in α-platelet granules and the biological activities thereof.

FIG. 2 shows the protocol for the production of PRP.

FIG. 3 shows the protocol for the production of PRGF.

FIG. 4 shows the protocol for the production of PRGF.

FIG. 5 shows the 2016 DEPA classification.

FIG. 6 shows cytokines and chemokines present in the colostrum isolate mixture and the biological activities thereof.

FIG. 7 shows the growth factors present in the colostrum isolate mixture and the biological activities thereof.

FIG. 8 shows the antibacterial and antiviral factors present in the colostrum isolate mixture and the biological activities thereof.

FIG. 9 shows the results of the experimentation for dental implantology (9A Table, 9B Graph).

FIG. 10 shows a diagrammatic image of an osteochondral lesion to the femoral condyle and subsequent filling with a matrigel bead.

FIG. 11 shows the histological scoring system according to O'Driscoll et al. (O'Driscoll et al., 1986).

The tables in FIGS. 12, 13 and 14 show the clinical trial data for the treatment of osteoarthritis.

FIG. 15 shows the Radiological Union Score system.

FIG. 16 shows the results of the experimentation for the management of fractures and nonunions.

FIG. 17 shows the lameness degree scores according to the criteria of the American Association for Equine Practitioners.

FIG. 18 shows the results of the experimentation for increasing the post-surgery healing process of tendons and ligaments, while FIG. 19 compares the results obtained in the clinical trials of each product tested and shows the results of the statistical analysis.

FIG. 20 shows the results of the tests for the treatment of muscle injuries and FIG. 21 shows the results of the experimentation for each group of animals.

FIGS. 22, 23, 24 and 25 show the results of the tests for the treatment of alopecia.

FIGS. 26, 27 and 28 show the results of the tests for skin rejuvenation and acne treatment.

FIG. 29 shows the Wound Bed Score by Falanga 2006, used for staging the skin lesions in subjects subjected to the experimental protocol.

FIG. 30 shows the results after related treatment for the treatment of burns, the treatment of skin wounds and ulcers and the treatment of keloids and hypertrophic scars and FIG. 31 shows the results of the statistical analysis.

FIGS. 32, 33 and 34 show the results of the treatment of phlebostatic ulcers.

FIGS. 35 to 43 show the results of the ophthalmological tests.

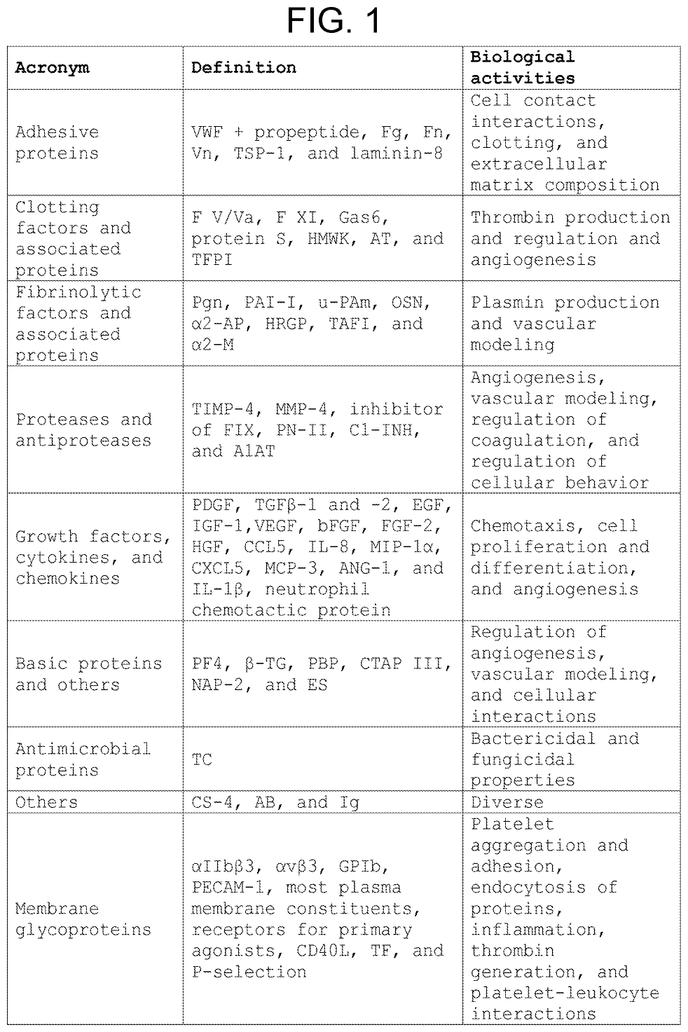

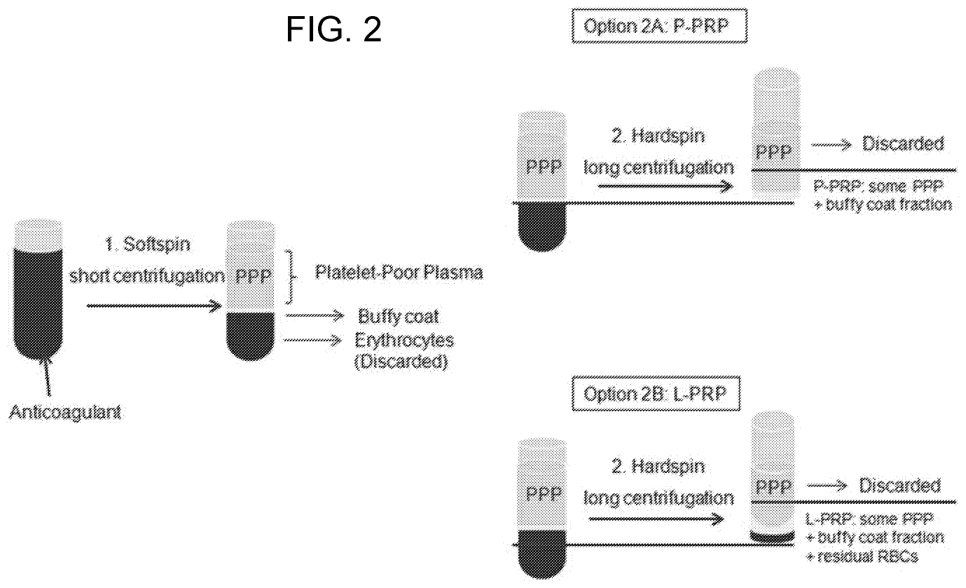

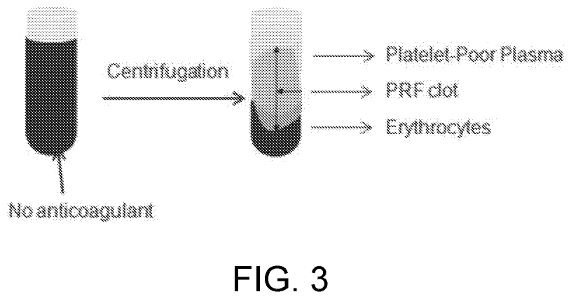

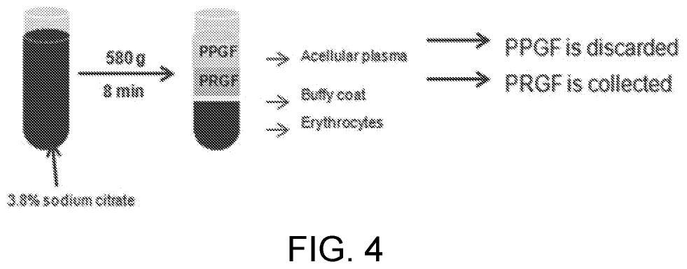

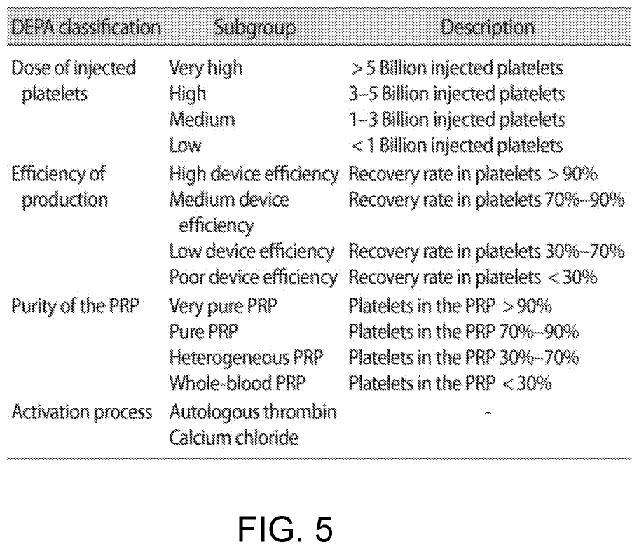

### DETAILED DESCRIPTION OF THE INVENTION

According to a first object of the invention, the composition described comprises platelet concentrates (PCs) and a colostrum isolate mixture, rich in biological factors.

In particular, such biological factors comprise: growth factors, cytokines, chemotactic factors, stem cell stimulating factors, complement proteins C3a/C4a, antibacterial and antiviral factors.

In particular, the colostrum isolate mixture comprises:

Lactoferrin and Transferrin

Lactoferrin and transferrin are among the main proteins involved in iron metabolism. Lactoferrin, in particular, has several other functions, including acting as a direct antimicrobial agent against a wide range of bacteria and viruses. Furthermore, lactoferrin has been shown to stimulate the growth of several cell lines in vitro, including fibroblasts and intestinal epithelial cells, suggesting that the presence thereof in colostrum may be important in regulating intestinal development in infants.

Cytokine

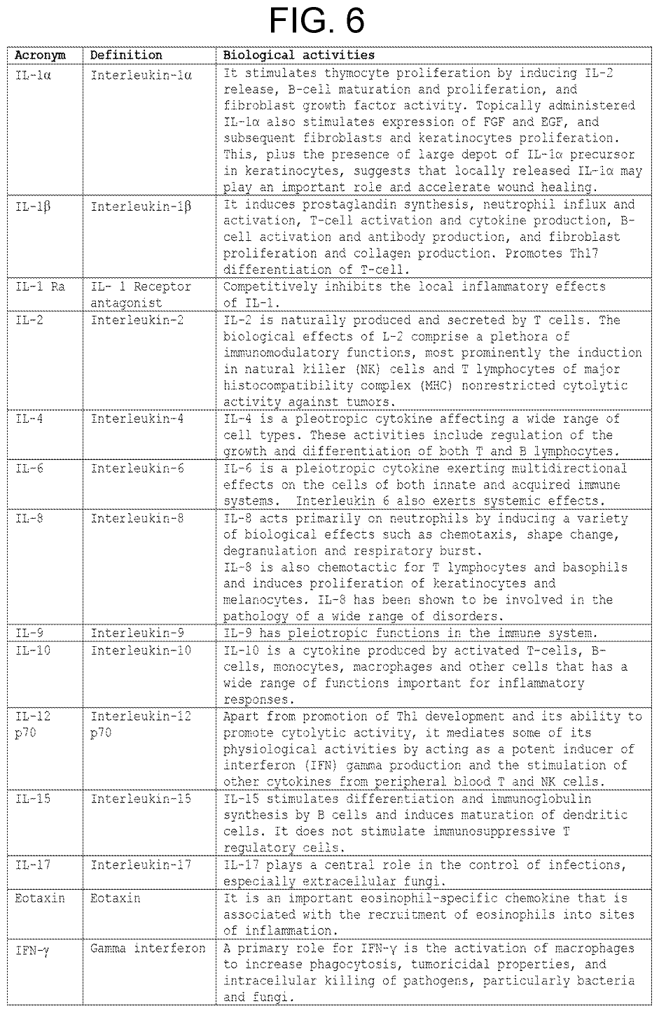

These are small soluble proteins, which act in an autocrine/paracrine manner by binding to specific cellular receptors, operating in networks and orchestrating immune system development and function. Cytokines act at picomolar concentrations. Experiments show that bovine colostrum can modulate the activity of human immune cells in vitro. Both cytokines linked to innate immunity, such as TNF-α, IL-1, MCP-1 and IL-15, and those linked to the regulation of acquired immunity, such as IL-2, IL-4, IFN-γ, IL-9, and IL-17, have been highlighted as important components of the colostrum isolate mixture. Among the cytokines identified, IL-17, which is the distinctive cytokine for T helper 17 (Th17) cells, is present in high concentrations in the mixture. Interleukin 17 plays a fundamental role against bacterial and fungal infections and has a relevant proinflammatory activity. It has recently been shown that IL-17 has a regulatory role in hematopoiesis, being involved in the modulation of both hematopoietic and mesenchymal stem cells. High levels of IL-15 were also measured, which induces the proliferation of T, B and natural killer (NK) cells, stimulating the maturation of the immune system and also possesses a unique antitumor activity. Interestingly, both proinflammatory stimulation (IL-1, TNF-α, IFN-γ and IL-12) and inhibitory anti-inflammatory cytokines (IL-10, IL-1Ra and TGF-β) are present in the colostrum isolate mixture of biological factors. Therefore, the use of such a balanced product, both in vitro and in vivo, should ensure that excessive stimulation/inhibition is avoided with regard to the delicate regulation of immune responses. The main cytokines and chemokines present in the mixture are shown in FIG. 6.

Growth Factors

Growth factors are so called because they have historically been identified for the ability thereof to stimulate the growth of cell lines in vitro. In reality, these peptide molecules possess multiple and remarkably diversified functions. The main effects, which derive therefrom are represented by the construction, maintenance and repair of bones, muscles, nerves and cartilage, the stimulation of fat metabolism, regulation of protein metabolism, maintenance of proper blood sugar levels, regulation of substances responsible for mood control and tissue healing. A further effect linked to the presence of growth factors seems to be due to the anti-aging action thereof, particularly carried out on the skin.

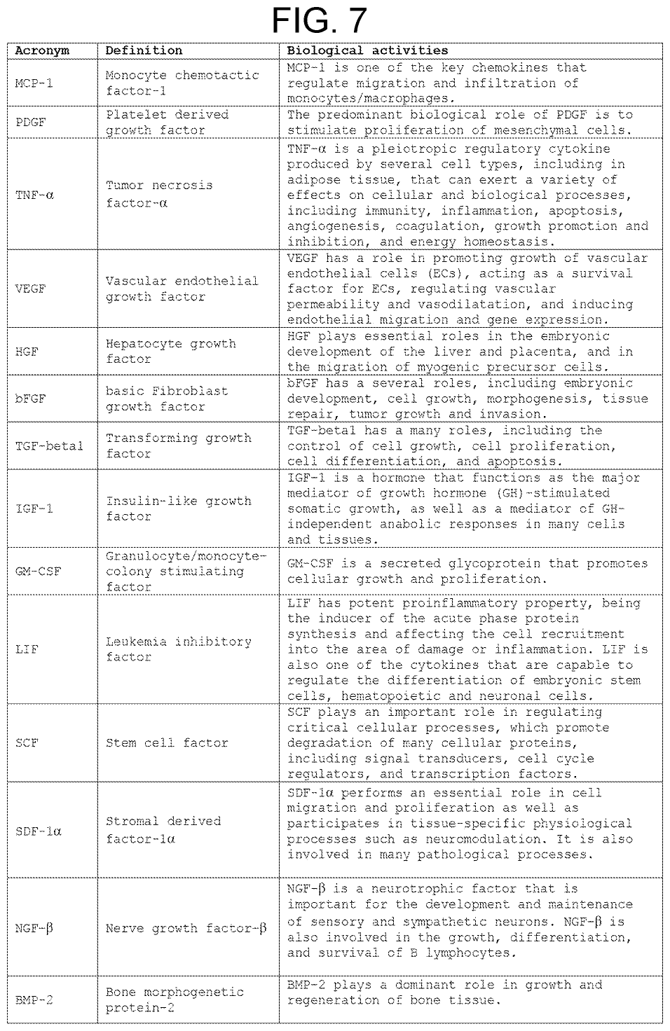

The main growth factors present in the mixture are shown in FIG. 7.

Granulocyte Colony Stimulating Factor (G-CSF) and Granulocyte-Macrophage Colony-Stimulating Factor (GM-CSF).

These are key substances for the proliferation, maturation, and expansion of the progenitor cells of bone marrow, and the role thereof as stimulating factors for stem cells is now well defined.

Other Components

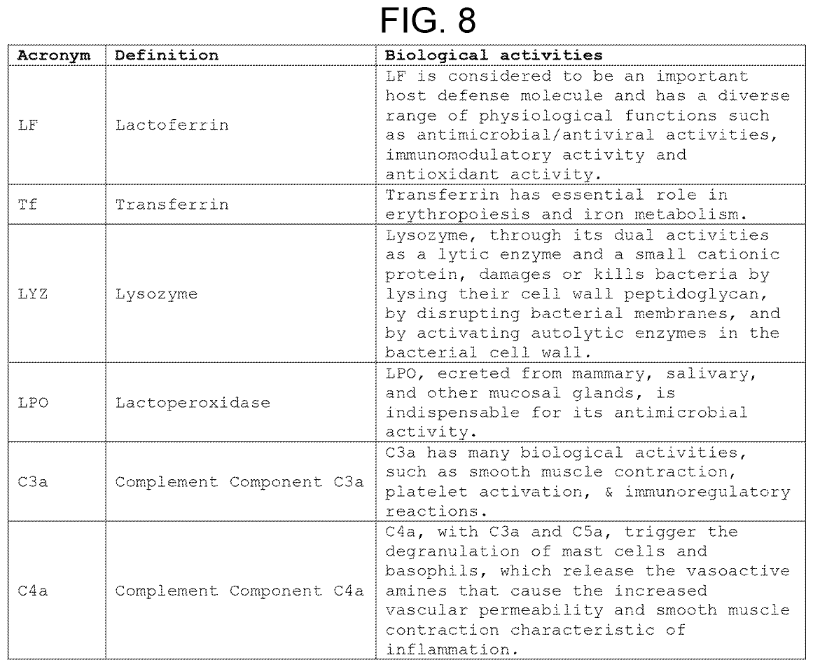

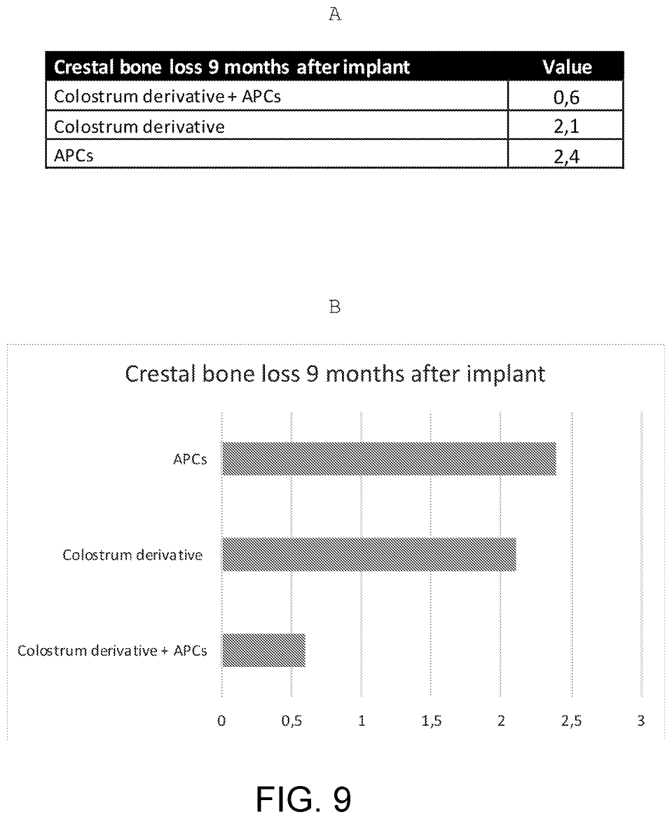

The mixture also contains other antibacterial and antiviral factors (FIG. 8), APLN peptides (not properly recognized as growth factors but with functions similar to proteins belonging to this class), non-peptide trophic factors and vitamins, in particular vitamin A, vitamin E, vitamin B12, but also traces of other vitamins, such as D and provitamin A (beta carotene).

In a preferred aspect, the colostrum used for the purposes of the present invention is obtained from humans or animals and, preferably, is obtained from cattle. In fact, colostrum from herbivores has been found to contain several growth factors and cytokines to a greater extent.

Bovine colostrum is the richest source of biological factors and therefore the most advantageous. In particular, dairy breeds have been shown to produce colostrum with the highest concentration of cytokines, growth factors, chemotactic factors, stem cell stimulating factors, complement proteins, antibacterial and antiviral factors, immunoglobulins. Taking the immunoglobulin concentration as an indicator of colostrum quality, the breed which produces the colostrum richest in bioactive factors is Jersey with 9.0% immunoglobulins, followed by Ayrshire with 8.1%, Brown swiss with 6.6%, Guernsey with 6.3% and Friesian (Holstein) with 5.6%.

These biological factors are highly conserved in phylogenesis and therefore have a considerable cross-reaction activity between the various species, therefore it is possible to use factors isolated from other mammals such as cattle, horses, camelids, etc. on humans.

The colostrum used for the purposes of the present invention is preferably collected before the newborn has had the opportunity to nurse and empty the udder of the first fraction produced, regardless of the time elapsed from the moment of birth. In fact, the greatest concentration of active ingredients is present precisely in the first fraction of colostrum produced by the udder. In a preferred aspect, the cows are preferably at the second or third birth.

Advantageously, the method for preparing the colostrum mixture rich in biological factors does not include steps or operations, which could damage such biological factors, reducing the activity thereof.

For example, no heat sterilization steps are carried out which can lead to the loss of large amounts of biological factors in the final product following the degradation caused by high temperatures.

Or, the separation of casein, necessary as it can lead to allergic reactions, cannot include a coagulation/precipitation step, followed by filtration, at low pH values which can lead to the denaturation of many proteins, including various biological factors present in the colostrum.

Consequently, for the preparation of the colostrum isolate mixture rich in biological factors according to the present invention, extraction processes will be used which allow obtaining a mixture of factors in a biologically active form.

Processes which can be used for preparing the mixture rich in biological factors are described for example by P. Sacerdote et al. (“Biological components in a standardized derivative of bovine colostrum”—Journal of Dairy Science, Volume 96, Issue 3, 1 Mar. 2013, pages 1745-1754) and in international patent application WO2011064114, which describes a process for obtaining a colostrum isolate mixture of biological factors based on an initial microfiltration step with a membrane with molecular porosity between 2-6 microns, a passage for collecting the permeate containing the biological principles, an optional sterilizing filtration step of the permeate and an optional removal step of the water contained in the permeate.

A similar colostrum extraction process is described in international patent application WO 2017/134559.

Other extraction processes may be used, as long as they meet the above requirements.

In a preferred aspect of the invention, the colostrum isolate mixture rich in biological factors is obtained according to the process described by P. Sacerdote et al., comprising the steps of:
1) dilution;
2) skimming;
3) ultrafiltration;
4) dialysis;
5) microfiltration.

In a preferred aspect of the invention, in step 1), a 100 kg amount of colostrum is placed in a reactor and diluted 1:10 with deionized water added with NaCl until reaching the concentration of 0.9%.

In another preferred aspect of the invention, in step 2) of skimming, the whole mass is centrifuged at 12,400 g at a temperature of 20-25° C. to remove the fat part.

According to another preferred aspect of the invention, in step 3), the previously obtained liquid phase is subjected to ultrafiltration through a membrane with 300 KDa cut-off, always maintaining the temperature of 20-25° C., to remove the large proteins and pathogenic microorganisms.

For example, the caseins are removed from among the large proteins.

In a further preferred aspect of the invention, in phase 4) of dialysis, the colostrum isolate mixture rich in biological factors thus obtained is then dialyzed with a 5 KDa membrane to remove any preservatives or drug residues present in the starting colostrum and especially the lactose in solution.

In another preferred aspect of the invention, in the microfiltration step 5), the product obtained is subjected to a series of sterilizing cross-flow filtration passages with 0.2 μm membranes and subsequently frozen.

Therefore, the product obtained is characterized by a mixture of biological factors, also comprising the immunoglobulins (IgG, IgA and IgM) in this step, which will subsequently be removed.

As the last step of the preparatory process, the mixture is subjected to a lyophilization step.

Thereby, a sterile, preservative-free, hypoallergenic powder is obtained (casein and lactalbumin are responsible for over 95% of bovine milk allergies) of very high solubility and with the highest possible concentration of active factors.

In a preferred aspect, a qualitative and quantitative control of the biological factors is included both in vitro and in vivo.

To evaluate the quality of the processes implemented to obtain a standardized final product, some key factors of colostrum are used as markers, such as lactoferrin and transferrin, which are present in high concentrations; furthermore, a search for a large number of bioactive factors can also be performed on the final product using known single ELISA tests.

In a particularly preferred aspect of the invention, the mixture derived from the colostrum is characterized, in the main components, by a content of:
Growth factors: from 600 to 1900 pg/mg, preferably from 650 to 1850;
Cytokines: from 45 to 300 pg/mg, preferably from 50 to 250;
Chemotactic factors: from 2 to 20 pg/mg; preferably from 3 to 15;
Stem cell stimulating factors: from 100 to 1200 pg/mg, preferably from 130 to 1100;
C3a/C4a complement proteins: from 1 to 5 pg/mg, preferably from 1.20 to 3;
Antibacterial/antiviral factors: from 20 to 80 μg/mg, preferably from 22 to 70.

To obtain a mixture of active factors isolated from colostrum which can be used in injectable form in those countries where health legislation prohibits the injection administration of heterologous immunoglobulins, it is necessary to add further steps to the method described above which allow obtaining a product deprived of the immunoglobulins. The product thus obtained has a high activity of biological factors because the content of active factors is much higher at the same weight; in fact, the immunoglobulins which do not perform a regenerative activity on cells and tissues would represent 30-40% of the mixture if not eliminated.

According to a particularly preferred aspect of the present invention, the process for preparing the mixture of active factors isolated from the colostrum thus comprises a further purification step.

In particular, such a step is carried out on the concentrated product before freezing and lyophilization, and is characterized by an IgG and IgA depletion step, which, in a preferred aspect of the invention, is carried out by means of affinity chromatography.

The colostrum purification step further comprises a step of IgM depletion, for example carried out by tangential filtration or cartridge filters.

METHOD: In a preferred aspect in the method of the present invention, affinity chromatography is used by means of:
1. CaptureSelect™ IgG-Fc (ms) Affinity Matrix (Thermo Fisher Scientific) or equivalent product. This system has been specifically designed to purify IgG from different species (human, mouse, rat, rabbit, cow, horse, and sheep) by high affinity binding to the Fc region of IgG.
2. CaptureSelect™ Bovine IgA Affinity Matrix (Thermo Fisher Scientific) or equivalent product. This system has been specifically designed for the purification of bovine IgA from whey/colostrum. In CaptureSelect™ Bovine IgA Affinity Matrix, affinity ligands are created using proprietary technology based on single domain antibody fragments derived from camelids. The ligand is a 13 kDa single domain fragment comprising the 3 complementarity determining regions (CDRs) which form the antigen binding domain, effectively produced by the yeast Saccharomyces cerevisiae in a process devoid of animal components.

In a preferred aspect of the present invention, the depletion of IgM and immunoglobulin aggregates occurs by means of a tangential flow filtration step, which uses membranes with a molecular cut-off of about 750 kDa/0.02 μm, or by using cartridge filters with pores of the same size. The IgM depletion is performed by ultrafiltration since such molecular species are characterized by a much higher molecular weight with respect to the bioactive component. More in detail, the pentamers of the M immunoglobulins, which have an average molecular weight of 800-900 kDa, can be removed by tangential filtration, or cartridge filters, using membranes of appropriate size, while the proteins with a lower molecular weight can cross them without being retained. Different filtration methods were tested as well as a variety of filters and the best results in terms of IgM depletion and aggregates were obtained using cartridge filters with pores of 750 kDa/0.02 μm, type ENTEGRIS Savana® PS cartridge filter 0.02 micron or equivalent product.

In a second particularly preferred aspect, exclusively for the injective use of the product, a further purification step of the colostrum isolate mixture is necessary to deprive it of the possible presence of bacterial endotoxins (exogenous pyrogens).

In a preferred aspect of the present invention, the removal of the endotoxins occurs by means of specific kits, which use an affinity chromatography, such as the Bio-Rad Proteus Endotoxin Removal kits or equivalent products. Alternatively, cartridge filters with directly incorporated positively charged nylon membrane can be used which, by strongly interacting with the negatively charged endotoxins, can adsorb consistent concentrations of the latter present in solution. The best results in terms of endotoxin removal were obtained using BEA Technologies POSINYL nylon 6.6 membrane cartridge filters or equivalent product.

In a further preferred aspect of the present invention, the aforesaid concentrated sample is subjected to a lyophilization process.

The process was developed with a view to industrial application. All the process steps are transferable with a view to a large-scale purification process.

According to a preferred aspect of the present invention, in order to increase the bioavailability of the colostrum isolate mixture and to protect it from the action of enzymes or from denaturing environments, this can be included in a liposomal formulation, according to the techniques known in the prior art.

In the preparations of the invention, the two components can be present in the following amounts (in 5 mL of plasma):

| | |
|---|---|
| Platelet concentrates or PCs (understood as the number | 1 × 10⁶/μL |
| of platelets) | |
| Colostrum isolate mixture rich in biological | 10-1000 mg |
| factors | |

In a preferred aspect of the invention, the two components can be present in the following amounts:

| | |
|---|---|
| Platelet concentrates or PCs (understood as the number | 1 × 10⁶/μL |
| of platelets) | |
| Colostrum isolate mixture rich in biological | 25-750 mg |
| factors | |

For example, the two components can be present in the following amounts:

| | |
|---|---|
| Platelet concentrates or PCs (understood as the number | 1 × 10⁶/μL |
| of platelets) | |
| Colostrum isolate mixture rich in biological | 50-500 mg |
| factors | |

In a preferred aspect of the invention, the platelet concentrates are autologous.

According to the present invention, autologous platelet concentrates (APCs), together with the colostrum isolate mixture rich in biological factors, are used for the preparation of formulations.

In a preferred aspect, such formulations are represented by liquid solutions, lotions, foams, sprays, creams, salves, ointments, pastes, gels, membranes, clots, powders or other forms suitable for medical use.

Those skilled in the art are capable of identifying the additives, excipients and other components required to obtain such formulations, choosing them from the prior art.

In a preferred aspect of the present invention, the formulation forming the object thereof may contain other ingredients in effective concentration.

In particular, one or more of the following components may be included:

Hyaluronic Acid

Available in various forms (esters, salts, hydrolyzed, cross-linked, liposomal, etc.), among these sodium hyaluronate is preferred, the sodium salt thereof, in amounts between 5 and 50 mg/mL of plasma, preferably 25 mg/mL of plasma, for the abilities thereof to restore tissue tone and elasticity, as well as to improve and accelerate the re-epithelialization process.

Amino Acids

The main amino acids used by fibroblasts for the construction of collagen are Glycine, L-Proline, L-Leucine and L-Lysine. A mixture of these amino acids, in amounts between 10 and 50 mg/mL of plasma, preferably 20 mg/mL of plasma, stimulates the regeneration of collagen.

Hydroxyapatite

Hydroxyapatite (HA) is the main inorganic component of bone and forms 60-70% of the calcified skeleton and 98% of tooth enamel. It is biocompatible, binds quickly to adjacent hard and soft tissues and has a strong osteoconductive capacity. The clinical indications of hydroxyapatite concern the reconstruction of bone tissue and the lining of endo-osseous dental implants to promote osseointegration. Hydroxyapatite is also used as a component of skin fillers. In the case of bone reconstruction or dental implantology, the amounts used vary in relation to the surfaces to be reconstructed or covered. In the case of fillers, 1 mL of gel is used containing from 20 to 50% of hydroxyapatite microspheres in 5 mL of plasma, preferably 40% of microspheres.

β-Tricalcium Phosphate

Beta tricalcium phosphate is a polycrystalline bioceramic, with osteoconductive properties, which in contact with water releases hydroxyapatite crystals. It is used for the resolution of deep intraosseous periodontal defects (periodontal regeneration), for filling post-extraction bone cavities, for bone regeneration, etc. Beta tricalcium phosphate has a progressive resorption, unlike what occurs with hydroxyapatite, with a release of calcium and phosphorus ions which, for example in the case of intraosseous periodontal defects, contribute to the neo-apposition of bone, cement, and periodontal ligament. It is used in the amount of 1 g/mL of plasma.

According to an object of the present invention, the compositions and formulations detailed above are described for medical use.

In particular, these can be advantageously used for the treatment of conditions requiring tissue repair and regeneration in humans and animals.

In a preferred aspect, the compositions and formulations described are used in oral and maxillofacial regenerative surgical procedures (in the healing of post-extraction alveoli, in the treatment of periodontal defects, in endodontics and endodontic surgery, in maxillary sinus lift, in dental implantology); in orthopedics and sports medicine (in the treatment of tendon injuries, in the treatment of osteoarthritis, in the treatment of degenerative disease of the intervertebral discs, in the enhancement of the post-surgery healing process of tendons and ligaments, in the treatment of muscle injuries, in the management of fractures and nonunions); in dermatology and aesthetic medicine (in the treatment of vitiligo, in the treatment of acne, in the treatment of alopecia, in the treatment of stretch marks, in skin rejuvenation, in vaginal rejuvenation); of burns, in the treatment of wounds, in the treatment of skin ulcers, in the treatment of keloids and hypertrophic scars, in breast reconstruction); in ophthalmology (in the treatment of persistent corneal epithelial defects, in the treatment of dry eye syndrome, in the treatment of post laser-assisted in situ ocular surface keratomileusis or Laser-ASsisted In situ Keratomileusis—LASIK, in corneal surgery, in the treatment of chronic ocular hypotonia after glaucoma surgery; in veterinary medicine and surgery, for all the uses already described in human medicine and surgery.

The invention is described here in greater detail in the following experimental part.

### Example 1

### Topical Formulation (A)

| | Amount in |
|---|---|
| Component | 5 mL of plasma |
| Autologous platelet concentrates (APCs) | 1 × 10⁶/μL |
| (understood as the number of platelets) | |
| Colostrum isolate mixture | 500 mg |
| Hyaluronic acid | 125 mg |

The composition can be possibly modified by adding the following components (alternatively):

| | Amount in |
|---|---|
| Component | 5 mL of plasma |
| Hydroxyapatite | q.s. |
| β-tricalcium phosphate | 5 g |

### Example 2

### Injection Formulation (B)

| | Amount in |
|---|---|
| Component | 5 mL of plasma |
| Autologous platelet concentrates (APCs) (understood as | 1 × 10⁶/μL |
| the number of platelets) | |
| Colostrum isolate mixture | 50 mg |
| Hyaluronic acid | 125 mg |

The composition can be possibly modified by adding the following components:

| | Amount in | |
|---|---|---|
| Component | 5 mL of plasma | |
| Amino acids (mixture) | 100 | mg |
| Hydroxyapatite (40% microspheres gel) | 1 | mL |

### Example 3

### Clinical Trials

a) To evaluate the effectiveness of the preparations of the present invention in oral and maxillofacial regenerative surgical procedures, the clinical trials performed concerned dental implantology. In fact, as regards the healing of post-extraction alveoli, the treatment of periodontal defects, endodontics and endodontic surgery and maxillary sinus lift, the effectiveness of the preparations of the present invention is demonstrated by the homologous experiments performed for orthopedics and for plastic and reconstructive surgery.

Clinical Trials Performed for Dental Implantology:

Tooth replacement with a dental implant has led to a major revolution in the modern dental clinic. This technique is based on the osseointegration process, which allows a firm anchorage of titanium implant screws to living bone.

Alveolar bone loss around dental implants is detected in 5-10% of patients. A dental implant is considered a failure if it is lost, mobile or shows peri-implant bone loss greater than 1.0 mm in the first year and greater than 0.2 mm the following year.

The long-term clinical success of dental implants depends primarily on the preservation of the bone support around the implant, which is usually evaluated with radiographic images.

To evaluate the effectiveness of the compositions of the present patent in dental implantology, 21 patients were recruited, in the age group 30-60 years, who needed the replacement of a missing tooth in the posterior region of the mandible. The 21 patients were divided into three homogeneous groups of 7 subjects each (A, B and C).

Each individual of group A received a dental implant in the edentulous site filled with the Topical formulation (A) shown in EXAMPLE 1 (with β-tricalcium phosphate instead of hyaluronic acid); each individual of group B received a dental implant in the edentulous site filled with the same Formulation of group A but not containing APCs (thus containing only the colostrum isolate mixture and β-tricalcium phosphate); each individual of group C received a dental implant in the edentulous site filled with the same Formulation of group A but not containing the colostrum isolate mixture (thus containing only APCs and β-tricalcium phosphate).

Crestal bone loss was measured on the mesial, distal, buccal, and lingual side of each implant by periapical radiographs 9 months after implant placement.

The group A implants were 100% successful with crestal bone loss averages far below the 1.0 mm cutoff within the first year of implantation.

Conversely, the mean crestal bone loss 9 months after implant placement in Group B and Group C was 2.1 mm and 2.4 mm respectively, indicating a certain percentage of failure.

FIG. 9 shows the results of the experimentation for each group of individuals (9A Table, 9B Graph).

b) To evaluate the effectiveness of the preparations of the present invention in orthopedics and sports medicine, the clinical trials performed concerned the treatment of osteoarthritis, the management of fractures and nonunions, the increase in the post-surgery healing process of tendons and ligaments and the treatment of muscle injuries (the treatment of tendon injuries and the treatment of degenerative disease of the intervertebral discs being included in the previous experiments).

Clinical Trials Performed for the Treatment of Osteoarthritis:

The most used animal model for cartilage research is sheep. This model provides advantages such as joint size, cartilage thickness and the possibility of performing procedures even in arthroscopy, it further has a limited capacity for spontaneous healing, consequently degenerative and traumatic lesions lead to an inevitable acceleration of the arthritic processes.

The joint of the sheep is larger than that of the dog and it is possible to make lesions greater than 6 mm, a dimension considered not capable of spontaneous healing.

The proportion between cartilage thickness and subchondral bone and the texture of the subchondral bone is more similar to that of humans, when compared to that of other animals.

In the evaluation of chondral lesions, this model allows studying the repair of partial or full thickness lesions, in fact the thickness of the medial femoral condyle varies from 0.8 to 2.0 mm. In addition, the joint size allows the creation of lesions similar to those of small size, but already clinically relevant, observed in humans.

To evaluate the effectiveness of the compositions of the present patent, 15 sheep of the same age and of similar size, of female sex, were divided into three homogeneous groups of 5 subjects each (group A, B and C).

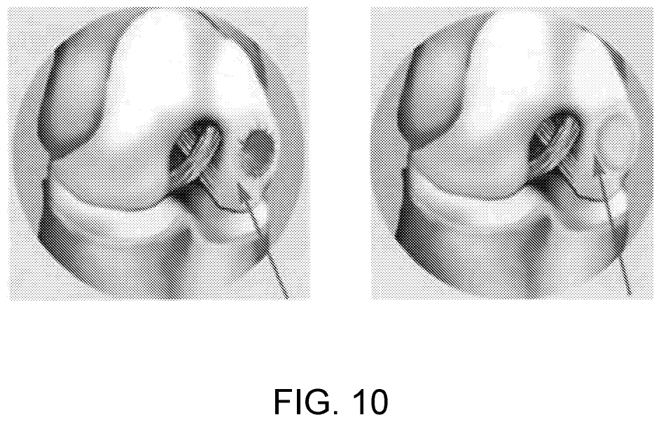

Under general anesthesia, through a traditional surgical access to the knee (open), a full-thickness lesion of 10-15 mm in diameter was made in the central portion of the right medial femoral condyle with special instruments. In the animals of group A, the defect was filled with a matrigel bead+the composition referred to as the “Injection formulation B” (shown in Example 2). In the second group (B), the defect was filled with a matrigel bead+the same injection formulation containing, however, only the colostrum isolate mixture and hyaluronic acid as the active ingredient. In the third group (C), the defect was filled with a matrigel bead+the same injection formulation containing, however, only APCs and hyaluronic acid as the active ingredient. Similarly, the contralateral knee was operated on and served as a negative control (matrigel only). FIG. 10 shows a diagrammatic image of an osteochondral lesion to the femoral condyle and subsequent filling with a matrigel bead.

After suturing and awakening, each animal was given painkillers and antibiotics for 5 days, and was allowed to stand immediately after surgery.

Four months after the surgery, the animals were sacrificed, proceeding with the removal of the operated femurs. The samples were then sent to the laboratory for the evaluation of cartilage repair.

An initial macroscopic evaluation was performed in the laboratory, taking into consideration the features of the femoral condyle surface and the continuity thereof with the surrounding tissue. The overall appearance of the joint was then evaluated to check for the presence of initial or overt osteoarthritis.

After macroscopic evaluation, the samples taken were fixed in a 10% solution of buffered formalin, decalcified, embedded in paraffin and microtome sliced to create 5-6 μm thick sections. The sections were stained with toluidine blue for cell morphological evaluation and with Safranin O and Sirius red to evaluate the proteoglycan matrix and the collagen in the matrix under a polarized microscope. Alcian blue staining was also used to evaluate proteoglycan production.

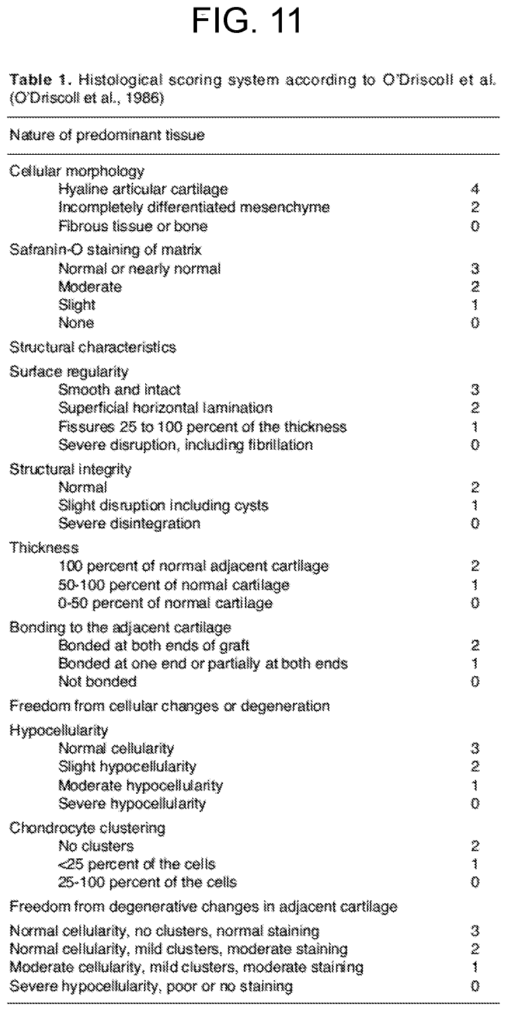

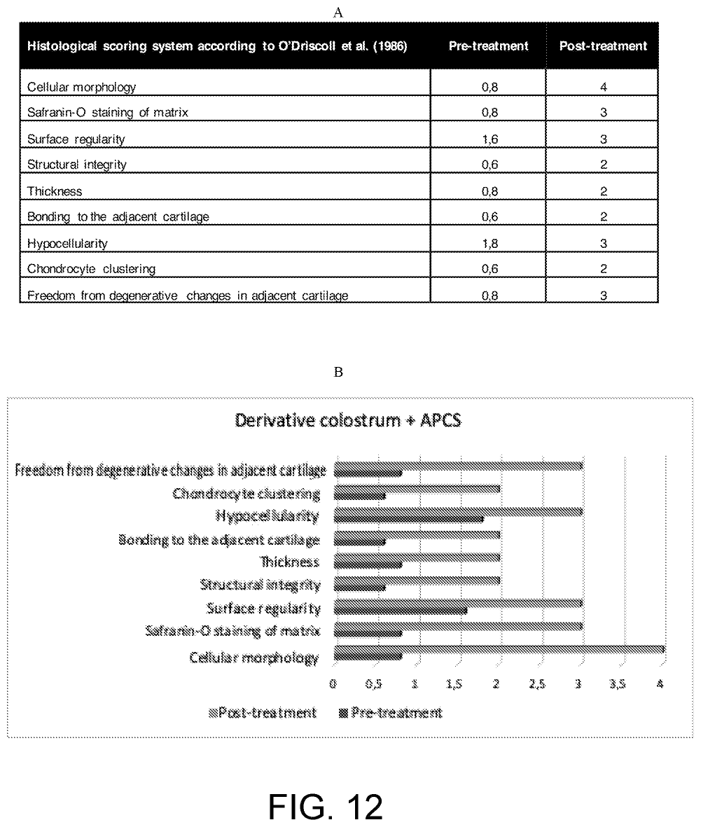

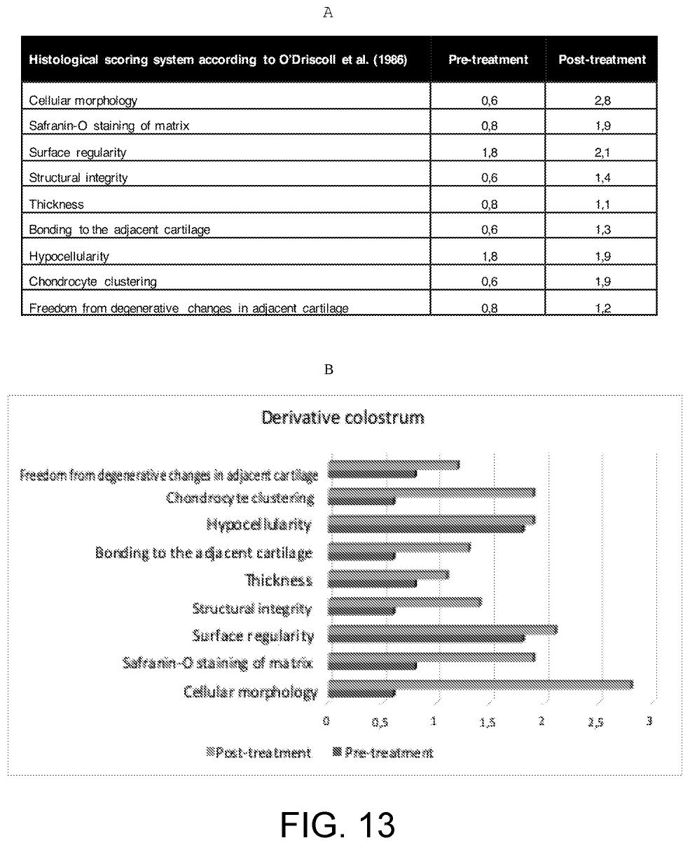

For the comparative evaluation of the results obtained with the different formulations, the histological scoring system according to O'Driscoll et al. was used (O'Driscoll et al., 1986) shown in FIG. 11.

The above controls showed that in the animals of group A, in which the defect had been filled with a matrigel bead+the composition referred to as the “Injection formulation B” (shown in Example 2), the osteochondral lesion to the femoral condyle was completely healed and the portion of cartilage removed had been restored, both from a macroscopic and microscopic (histological) point of view.

On the other hand, in the second group (B), in which the defect had been filled with a matrigel bead+the same injection formulation containing as the active ingredient only the colostrum isolate mixture and hyaluronic acid, the complete restitutio ad integrum of the lesion did not occur, as evidenced with the histological scoring system according to O'Driscoll et al., and even less in the third group (C), where the defect had been filled with a matrigel bead+the same injection formulation containing, however, only APCs and hyaluronic acid as active ingredients.

In most of the negative controls (contralateral knee, in which the defect was filled with a matrigel bead only), the lesion area was almost completely replaced by scar tissue.

The tables of FIGS. 12A, 13A and 14A show the averages of the scores assigned to each subject, before and after the relative treatment with one of the three tested products. The same data are plotted in FIGS. 12B, 13B and 14B.

Clinical Trials Performed for the Management of Fractures and Nonunions:

The evaluation tests were performed by choosing the dog as the reference species (Canis familiaris), both because the dog represents an excellent comparative study model for bone diseases, and for the ease of finding animals with spontaneous fractures.

For this study, 15 adult dogs with compound fracture of the femur following a road accident were divided into three groups (of 5 subjects each) homogeneous for age, body size and severity of the bone injury. During the fracture re-composition surgery, one of the three groups (A) was then treated with the composition referred to as the “Injection formulation B” (shown in Example 2) where hyaluronic acid was replaced with hydroxyapatite gel, the second group (B) was treated with the same hydroxyapatite gel but containing only the colostrum isolate mixture as the active ingredient, the third group (C) was treated with the same hydroxyapatite gel containing only APCs as the active ingredient.

Radiographic evaluations of the femur [anteroposterior (AP) and lateral radiographs] were performed immediately after the surgical procedure (week 0), at week 3, 6, 9, and 12, using standard radiological criteria for bone fracture healing (cortical callus formation, percentage of chondroid area and bone tissue, osteoblastic and fibroblastic activity, and formation of mature bone).

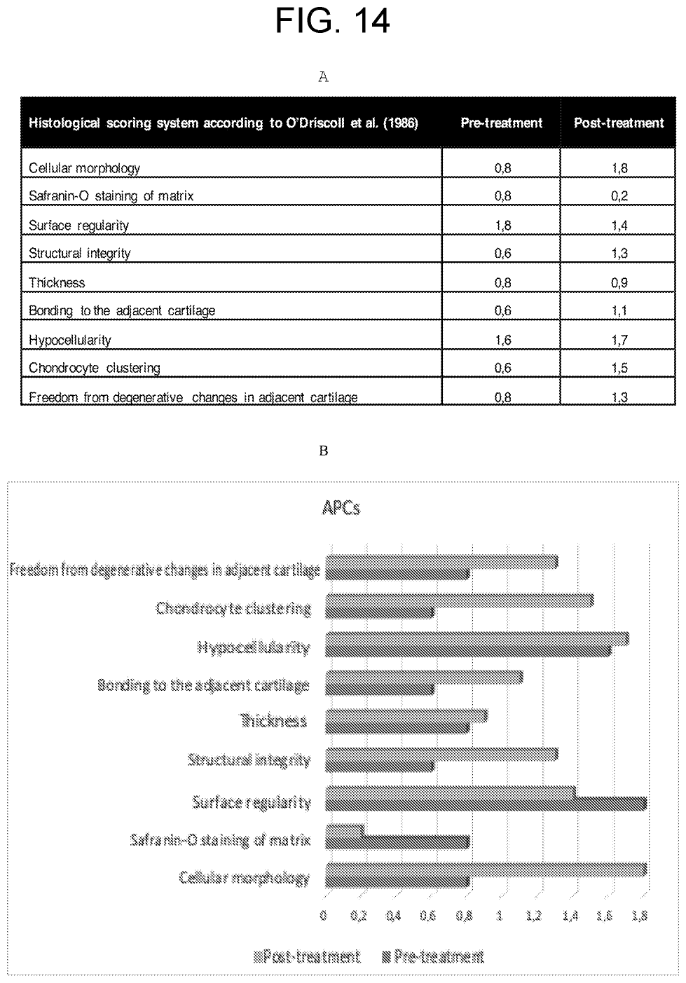

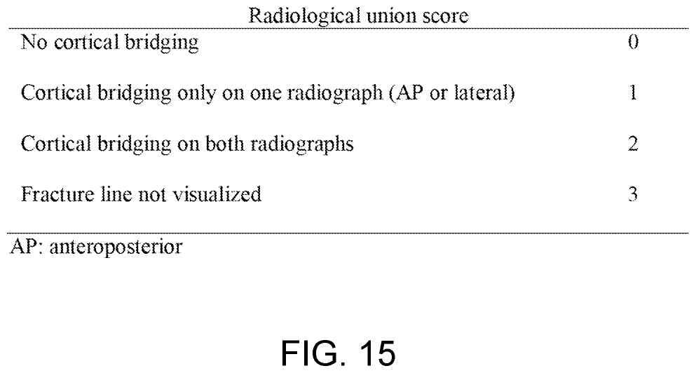

The statistical evaluation was performed with a scoring system, which includes the criteria shown in FIG. 15 (Radiological Union Score).

The union of the fracture with callus formation was obtained in all the dogs of group A, on the contrary in one animal of group B and in two of group C a pseudarthrosis (nonunion) was radiologically highlighted. In general, the control radiographs at weeks 3, 6, 9, and 12 showed delayed fracture union with less callus formation in groups B and C with respect to group A.

No adverse events were recorded in any of the treated groups.

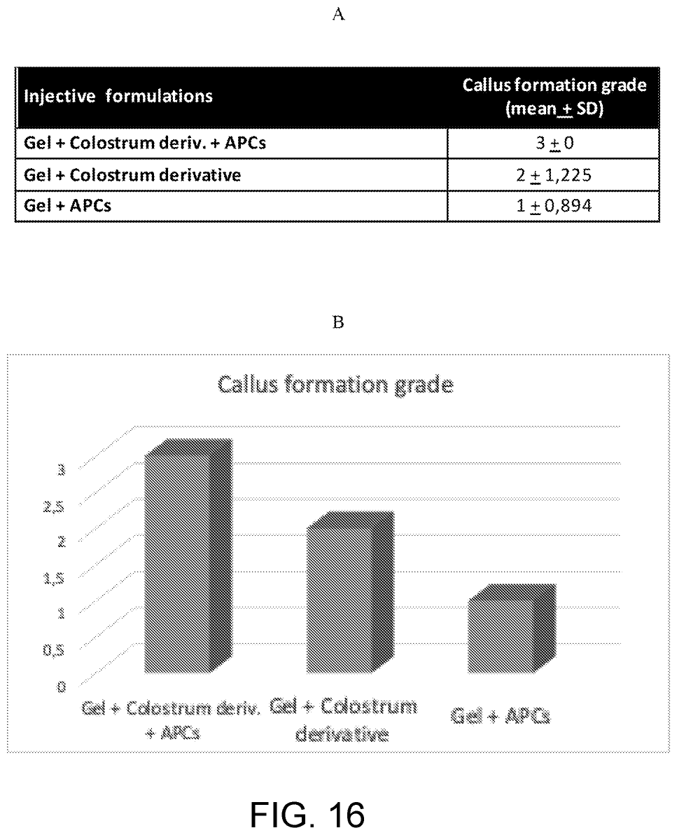

FIG. 16 shows the results of the experimentation for each group of animals (Table 16A, Graph 16B).

Clinical trials performed to increase the post-surgery healing process of tendons and ligaments:

In this case, the evaluation tests were performed by choosing the horse as the reference species (Equus caballus). In fact, competition horses are involved in particularly demanding activities, thus representing a highly significant model for the high mechanical overload typical of human athletes.

For this study, 15 competition horses with pre-existing tendon injuries were divided into three groups (of 5 subjects each) homogeneous for age, body size and injury severity. All had previously been treated with steroid-based drug therapies and non-steroidal anti-inflammatory drugs, with no improvement.

With the informed consent of their owners, one of the three groups (A) was then treated with the composition referred to as the “Injection formulation B” (shown in Example 2), the second group (B) was treated with the same injection formulation containing, however, only the colostrum isolate mixture and hyaluronic acid as the active ingredient, the third group (C) was treated with the same injection formulation containing, however, only the APCs and hyaluronic acid as the active ingredient.

For each group, the treatment consisted of a single injection per month of the respective formulation, for 5 consecutive months.

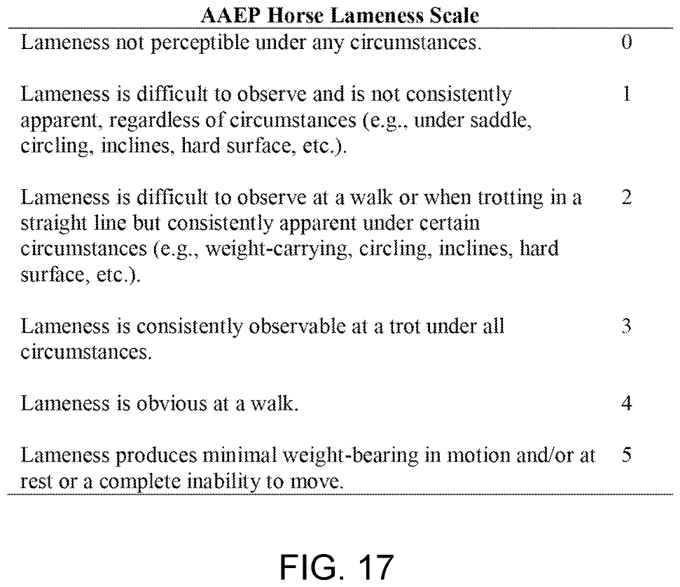

At month 0 (before starting the therapy), the horses were clinically examined (lameness degree, history of standing and moving, presence of pain, edema or swelling) and ultrasound to accurately locate the injury. The lameness degree score ranged from 0 to 5 according to the American Association for Equine Practitioners criteria (FIG. 17).

At month 6 (30 days after the last administration), the horses were reevaluated clinically and by ultrasound. Any adverse events and healing time were also recorded, i.e., the measure of the time needed to return to pre-injury activity levels, in addition to returning to competition.

None of the animals enrolled had major adverse reactions, either local or systemic, following the treatments.

All the horses in Group A showed marked improvement in their lameness degree, from an average of 1.4 (range, 1-2) to complete absence of symptoms at 6 months. All the animals in this group returned to pre-injury activity levels and, after 2-3 months of rehabilitation, were also able to return to competition. The ultrasound examination was also negative in all the animals.

The complete absence of symptoms at 6 months was observed in none of the horses in group B, ranging from an average of 1.2 (range, 1-2) to an average of 3.2 and the rehabilitation time to return to pre-injury activity levels were much longer. Furthermore, none of the subjects in this group was able to return to competitions.

Even in the horses of group C, the complete absence of symptoms was not observed at 6 months, passing from an average of 1.6 (range, 1-2) to an average of 2.6, moreover, despite the long rehabilitation times, none of these subjects returned to pre-injury activity levels.

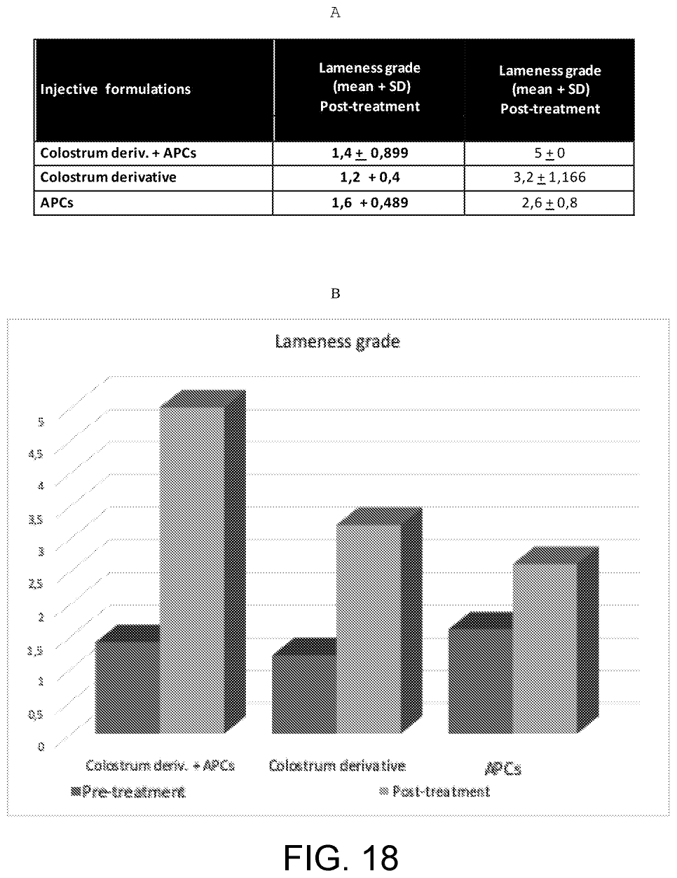

FIG. 18 shows the results of the experimentation for each group of animals (18A Table, 18B Graph), while FIG. 19 compares the results obtained in the clinical trials of each product tested and reports the results of the statistical analysis.

Clinical Trials Performed for the Treatment of Muscle Injuries:

Also in this case the evaluation tests were carried out by choosing the horse (Equus caballus) as the reference species.

18 horses with muscle tear injuries were divided into three groups (of 6 subjects each) homogeneous for age, body size and injury severity. Almost all of them had a history of trauma; in fact, most of the subjects had suffered the injury following accidental falls.

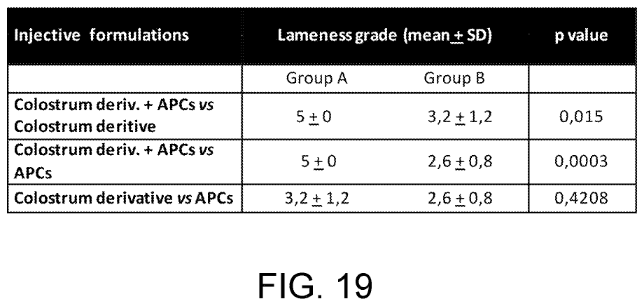

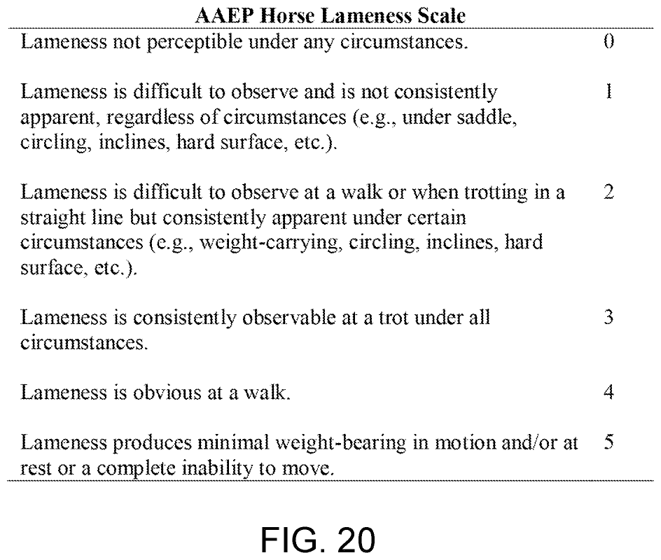

All the horses had palpable swelling at the muscle tear site and sensitivity to manipulation of the affected region. For all the subjects, the main symptomatology was further represented by varying lameness degrees (from 2/5 to 5/5 on the AAEP scale) (FIG. 20).

The ultrasound images showed the interruption of the normal pattern of the muscle fibers and the relative fascia for all subjects and the presence of an accumulation of intramuscular fluid, suggesting hematoma formation.

The affected forelimb muscles included: deltoid, cleidobrachialis, triceps, and brachial biceps. The injuries of the hind limbs involved the semimembranosus, the semitendinosus, the gracilis and the adductor muscles.

In all cases, radiographs were taken to rule out any potential underlying bone disease.

With the informed consent of their owners, one of the three groups (A) was then treated with the composition referred to as the “Injection formulation B” (shown in Example 2), the second group (B) was treated with the same injection formulation containing, however, only the colostrum isolate mixture and hyaluronic acid as the active ingredient, the third group (C) was treated with the same injection formulation containing, however, only the APCs and hyaluronic acid as the active ingredient.

For each group, the treatment consisted of a single ultrasound-guided injection of the respective formulation per week for 3 consecutive weeks.

At 30 days, the horses were reevaluated clinically and by ultrasound. Any adverse events and the healing time were also recorded, defined as the time taken by the patient to return to the previous work levels or to the time beyond which owners did not experience any further noticeable improvements.

None of the animals enrolled had major adverse reactions, either local or systemic, following the treatments.

All the horses in Group A had favorable outcomes, showing a marked improvement in their lameness degree, going from an average of 1.2 (range, 1-2) to a complete absence of symptoms and returning to an equal or higher activity level than before the injury. The ultrasound examination was also negative in all the animals.

The complete absence of symptoms at 30 days was observed in none of the horses in group B, passing from an average of 1.6 (range, 1-2) to an average of 3.8 and the rehabilitation times to return to pre-injury activity levels were much longer. Furthermore, the pattern of muscle fiber rupture remained apparent in all the follow-up images.

The complete absence of symptoms at 30 days was also not observed in the horses of group C, passing from an average of 1.4 (range, 1-2) to an average of 2.9, moreover 2 of these showed chronic lameness or stiffness which prevented them from returning to the same level of activity as before the injury. Also in this group, the muscle fiber rupture pattern remained apparent in all the follow-up images.

The outcomes between forelimb and hind limb muscle injuries were not significantly different.

FIG. 21 shows the results of the experimentation for each group of animals (Table 21A, Graph 21B).

c) To evaluate the efficacy of the preparations of the present invention in dermatology and aesthetic medicine, the clinical tests performed concerned the treatment of alopecia, skin rejuvenation and acne treatment. As regards the treatment of vitiligo, vaginal rejuvenation and the treatment of stretch marks, the effectiveness of the preparations of the present invention is demonstrated by the homologous experiments carried out for skin rejuvenation and for plastic and reconstructive surgery, as these lesions affect the skin layers.

Clinical Trials Performed for the Treatment of Alopecia:

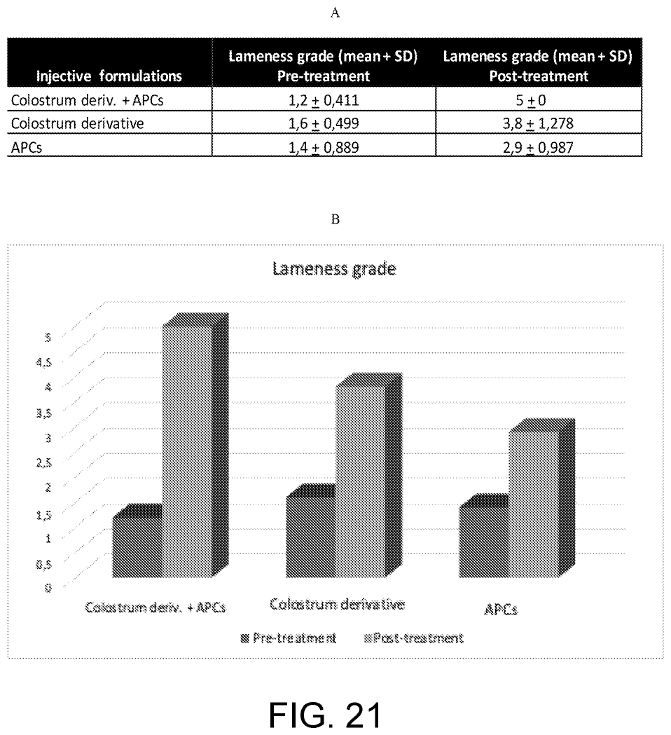

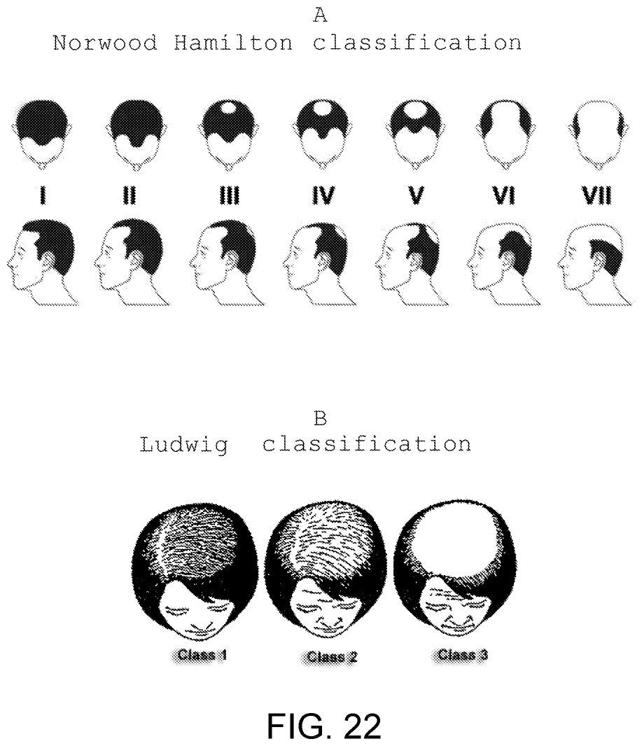

24 patients were recruited for this study, aged 19 to 73 years, 15 men who had stage IV androgenic alopecia according to Norwood Hamilton classification (FIG. 22A) and 9 women with stage I-III androgenic alopecia according to Ludwig's classification scale (FIG. 22B). The patients were divided into three homogeneous groups (A, B and C) consisting of 5 men and 3 women each.

One of the three groups (A) was then treated with the composition referred to as the “Injection formulation B” (shown in Example 2), the second group (B) was treated with the same injection formulation containing however, only the colostrum isolate mixture and hyaluronic acid as the active ingredient, the third group (C) was treated with the same injection formulation containing, however, only the APCs and hyaluronic acid as the active ingredient.

The respective formulations were injected into the scalp of patients in each group by interfollicular infusions (0.2 ml/cm²) at a depth of 5 mm, using a medical injection gun provided with a 30G needle. Three cycles of monthly injections (one cycle per month) were performed for three consecutive months.

The hair growth parameters, measured three months after the third injection by trichoscope examination, were compared with the measurements taken before the treatments. The results obtained indicate that three months after the third treatment in the three groups, the count and density/cm² hair growth significantly increased with respect to the baseline values especially in male patients and especially in those treated with “Injection Formulation B”.

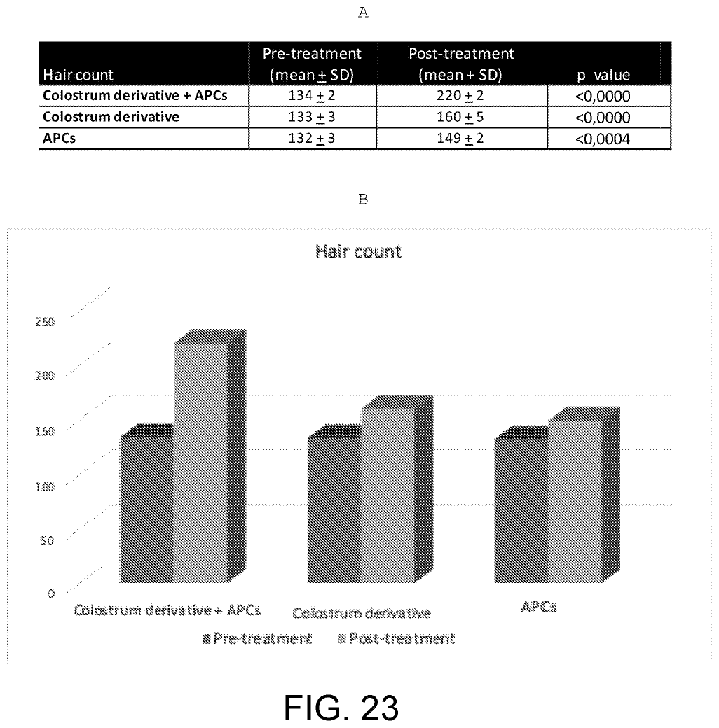

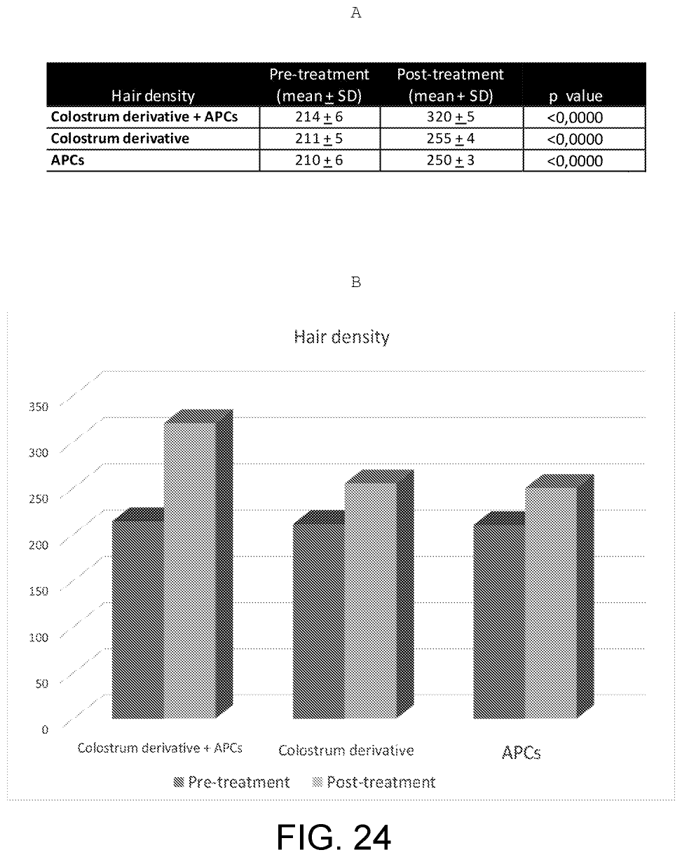

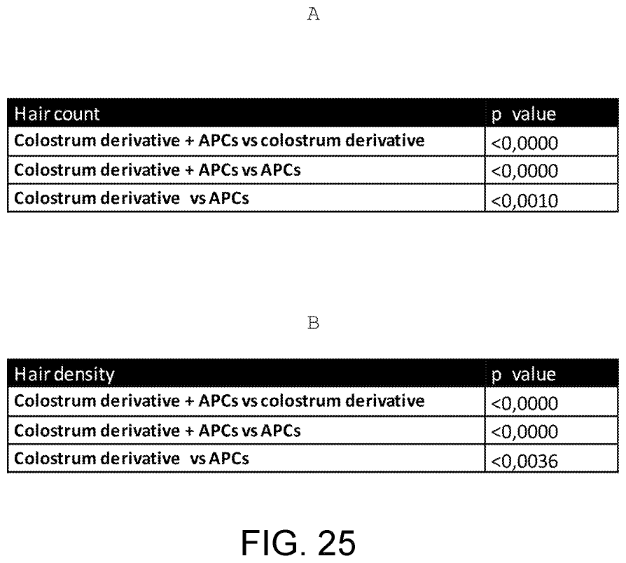

The patients in group A had a hair number of 134±2 and an average density of 214±6 per cm² before treatment, three months after the third treatment the hair number was 220±2 and the density was 320±5, a highly significant difference for both parameters (p<0.0000) (FIGS. 23A and 24A). Also in groups B and C, a significant increase was shown for both parameters analyzed (FIGS. 23A and 24A), but the hair number and the density/cm² were lower when compared to what was achieved in the patients treated with “Injection Formulation B”. The same results are plotted in FIGS. 23B and 24B, while FIGS. 25A and 25B compare the results obtained in the clinical trials by each product tested and the results of the statistical analysis.

Clinical Trials Performed for Skin Rejuvenation and Acne Treatment:

To evaluate the efficacy of the preparations of the present invention in facial rejuvenation and in the treatment of acne, 45 female patients, aged between 30 and 55 years, were divided into three homogeneous groups (A, B and C) of 15 patients each.

The most common problems of the patients before treatment included the presence of wrinkles, skin atrophy and facial sagging in the periorbital area and cheek region, active acne or scarring therefrom, patchy depigmentation and uneven skin texture.

One of the three groups (A) was then treated with the composition referred to as the “Injection formulation B” (shown in Example 2), the second group (B) was treated with the same injection formulation containing however, only the colostrum isolate mixture and hyaluronic acid as the active ingredient, the third group (C) was treated with the same injection formulation containing, however, only the APCs and hyaluronic acid as the active ingredient.

To reduce pain, local anesthetic cream and ice were applied to each patient prior to the procedure. To each group of patients, small aliquots of the respective formulation were infiltrated 5-6 mm deep, into the deep dermis and hypodermis, through hundreds of microholes, with a 30 G and 13 mm needle. At the end of the treatment, ice and an antibiotic ointment were applied topically to all patients.

The treatment was organized into “intense” and “maintenance” periods. In the intense treatment period, three consecutive treatments were performed 3-4 weeks apart. During the maintenance period, patients received up to five sessions at eight to ten week intervals.

At the end of the last treatment period, all the patients were asked to rate their satisfaction on a scale ranging from 0 to 3 (0=no improvement, 1 slight improvement, 2=moderate improvement, and 3=good improvement) for general satisfaction, for skin texture, for skin relaxation and firmness and for skin pigmentation. Furthermore, using the same rating scale, three independent doctors assessed the overall aesthetic improvement by comparing the digital photographs taken before and after the treatments.

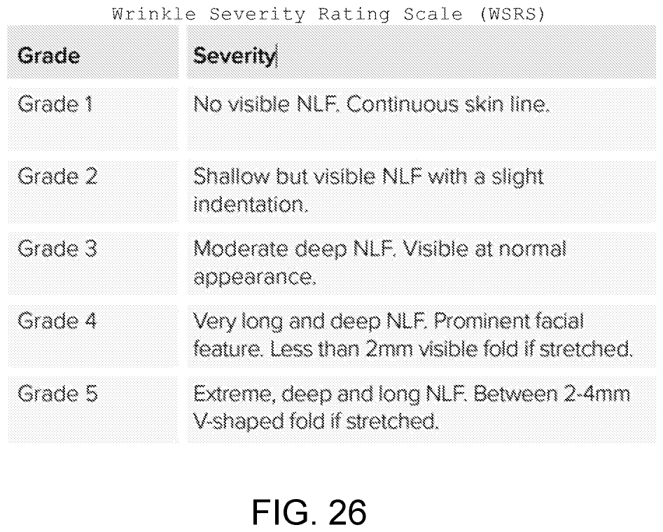

The Wrinkle Severity Rating Scale (WSRS) shown in FIG. 26 was also used.

For the statistical analysis, a P value<0.05 was considered statistically significant.

For all groups, no significant or persistent side effects were observed during the therapies, other than a mild and transient facial edema.

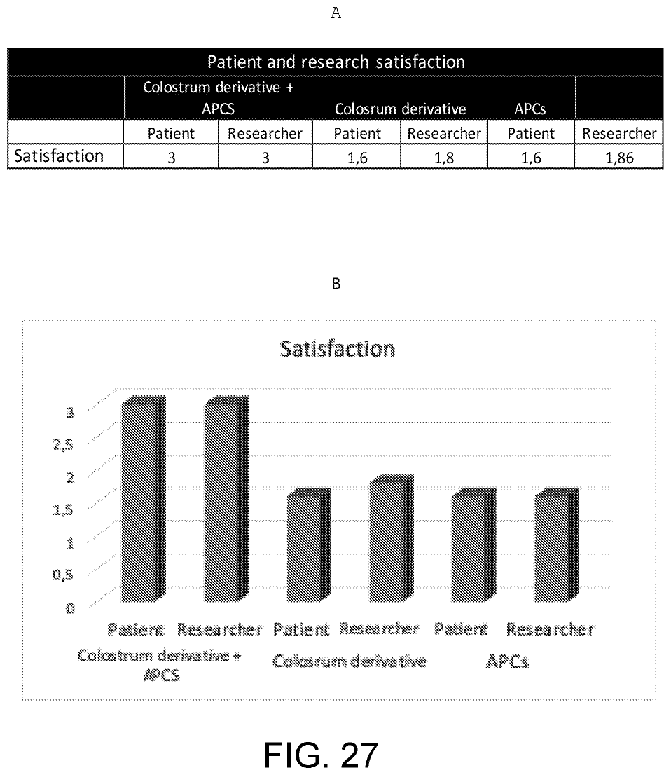

As regards group A, already during the intense treatment period a significant improvement was noted in the general appearance, in skin relaxation and turgor, in skin texture, both according to the evaluation scale of the patients and according to that of the doctors (FIGS. 27A and 27B). Pigmentation responded more favorably and earlier in the fair-skinned individuals. There was also a statistically significant increase in skin hydration, a minimization of the pores of the skin, which was shinier and brighter. A rapid regression of active acne and a significant reduction in the scarring therefrom were also apparent. The disappearance of fine wrinkles and a significant improvement in skin tone were recorded, especially in the periorbital skin and the contour of the lower eyelids. Furthermore, a certain regression of bags and sagging under the eyes was apparent.

Later with the therapy, again among the patients of group A, by virtue of the thickening and hydration of the dermis, the dry and parchment-like skin due to photoaging gradually recovered and the disappearance of skin telangiectasias was observed. The deep wrinkles of the upper lip, cheeks and nasolabial folds were also mostly gone.

As for the patients of group B and group C, on the other hand, in the initial stages of treatment, only the disappearance of the finest wrinkles and an increase in the degree of skin hydration were observed. Only in the most advanced stages of therapy did deep wrinkles show minimal attenuation, while no significant effect was had on active acne and the scarring therefrom.

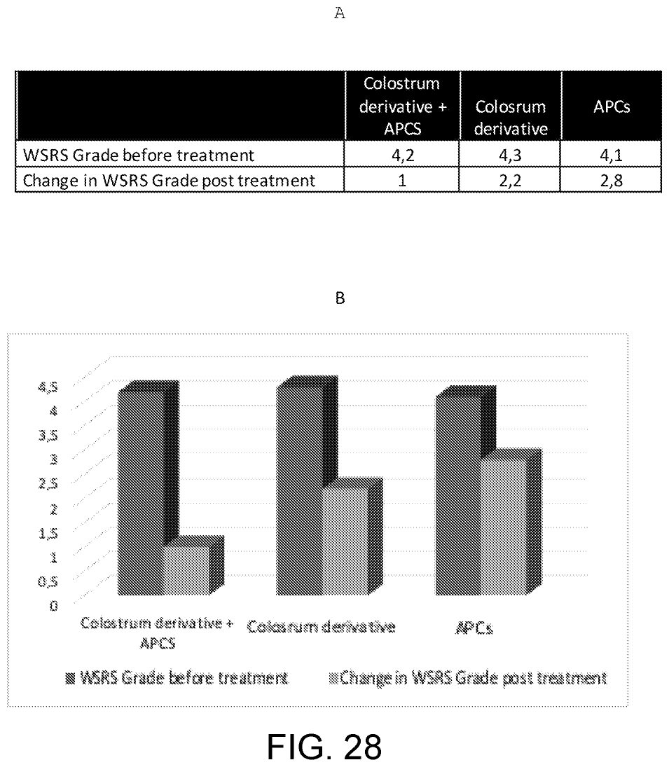

FIGS. 28A (Table) and 28B (Graph) show the results of the experimentation for each group of individuals.

d) To evaluate the efficacy of the preparations of the present invention in plastic and reconstructive surgery, the clinical tests performed concerned the treatment of burns, the treatment of skin wounds and ulcers and the treatment of keloids and hypertrophic scars. As regards breast reconstruction, the effectiveness of the preparations of the present invention is demonstrated by the homologous experiments carried out for skin rejuvenation and for the treatment of muscle injuries.

Clinical Trials Performed for the Treatment of Burns, the Treatment of Skin Wounds and Ulcers and the Treatment of Keloids and Hypertrophic Scars:

For the majority of skin lesions (wounds, burns, ulcers and sores) the evaluative tests were performed by choosing the dog as the reference species (Canis familiaris), both to test the compound in the veterinary field, and to evaluate the effects thereof in human dermatology, considering that the dog represents the best model of comparative study for skin diseases, largely sharing the etiology and pathogenesis with humans and the related symptomatology.

It was preferred to subject pets with spontaneous pathologies to therapy rather than laboratory animals with experimentally induced lesions, both for ethical reasons and for the poor correspondence between spontaneous lesions (especially as regards etiopathogenetic mechanisms) and experimentally induced lesions.

The clinical tests were performed at authorized veterinary facilities and with the informed consent of the owners of the animals, on 30 Canis familiaris subjects with various types of skin wounds, divided into 3 groups of 10 subjects each (A, B and C) homogeneous for age, sex, race, etiology, and wound severity. In each group one third of the animals, carriers of the above-mentioned skin pathologies, were also diabetic subjects.

Animals were specifically chosen with fairly serious skin solutions of different types and etiologies, from lacerated bruised wounds, to deep ulcers, burns, lesions caused by severe forms of dermatitis, to ulcers in the diabetic patient.

All the animals covered by this study were subjected to laboratory tests and specialist visits to exclude the presence of other diseases which could interfere with the therapeutic protocol (especially ectoparasitic infestations and dysendocrinies of the thyroid and hypothalamus-pituitary-adrenal axis), to carry out the staging of the skin wounds (essential for forming homogeneous groups) and to verify the suitability of the subjects recruited for inclusion in the therapeutic protocol. All the skin wounds were thoroughly documented, before and after therapy.

One of the three groups (A) was then treated with the composition referred to as the “Topical Formulation A” (shown in Example 1) in the form of a dermatological cream, the second group (B) was treated with the same dermatological cream containing, however, only the colostrum isolate mixture and hyaluronic acid as the active ingredient, the third group (C) was treated with the same dermatological cream containing only APCs and hyaluronic acid as the active ingredient. For each group, the treatment involved a single daily topical application of the respective composition until the skin wounds healed, where the transformation of the granulation tissue into scar tissue and the re-epithelialization of the wound surface was considered for healing. No concomitant local or systemic drug therapy was administered to the treated animals. During the entire treatment period, regular specialist dermatological examinations were performed to check the progress of the skin wounds.

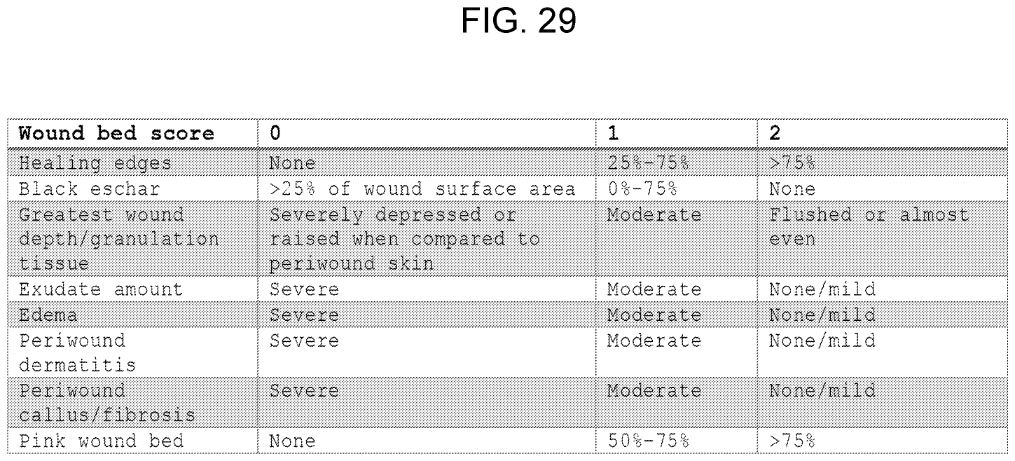

FIG. 29 shows the Wound Bed Score (best score 16; worst score 0) by Falanga 2006, used for staging the skin wounds in the subjects subjected to the experimental protocol.

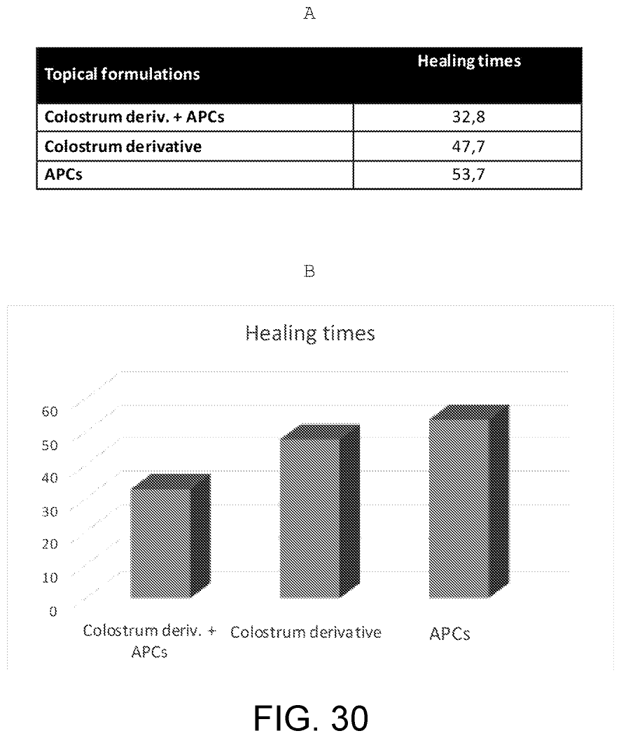

The table in FIG. 30A shows the average healing times after the relative treatment with one of the three products tested for each group. The same data are plotted in FIG. 30B.

As can be seen from the data shown in the figures, the healing times were significantly faster in the group treated with the composition of the present patent, with respect to the other two reference products.

To compare the results obtained from the clinical trials, Student's t (p<0.05 significant values; p<0.01 highly significant values) was used. The statistical calculations show that the difference in the post-treatment averages between the composition of the present patent and the other two reference products is highly significant. FIG. 31 shows the results of the statistical analysis.

The above demonstrates the greater efficacy of the composition based on autologous platelet concentrates and a colostrum isolate mixture of biological factors of the present invention, with respect to the two reference products.

In fact, the compound of the present invention has allowed obtaining much more rapid healing of all skin continuum solutions, with complete wound healing and re-epithelialization. Furthermore, it is important to underline that in none of the subjects treated with the composition of the present patent are the hypertrophic or keloid scars residual, one of the most common wound healing disorders, contrary to what was observed in the subjects treated with the two reference products. It should be noted that in many cases the treatment with the formulation of the present invention has also led to the regeneration of adipocytes and hair follicles, making the new tissues indistinguishable from normal skin, probably by virtue of the combined action of growth factors and cytokines. Furthermore, the local treatment with the composition of the present invention did not give rise to any side effects, thus proving not only effective but also safe.

As instead regards the treatment of phlebostatic ulcers, clinical trials were conducted on humans, recruiting 10 patients with advanced trophic leg ulcers. The patients who arrived in the clinic underwent echo-color Doppler and disease classification (venous, arterial, mixed) and were then subjected to home medical therapy with the composition referred to as the “Topical Formulation A” (shown in Example 1) in the form of a dermatological cream to be applied on the ulcer base in association with a disinfection protocol of the ulcers themselves (cleansing of the ulcer base and debridement) and the use of an elastic compression bandage.

The dressings were initially performed twice a week and then on a weekly basis. The tissue healing occurred from a minimum of 2 months to a maximum of 6 months of treatment.

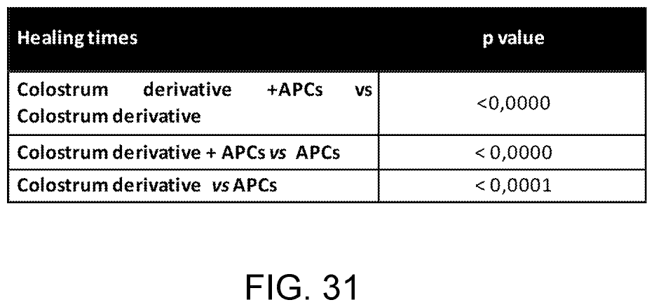

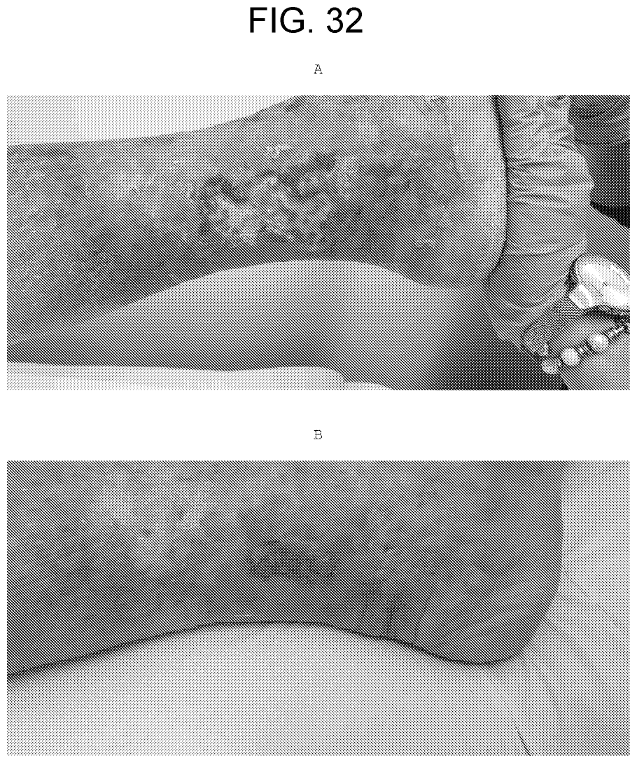

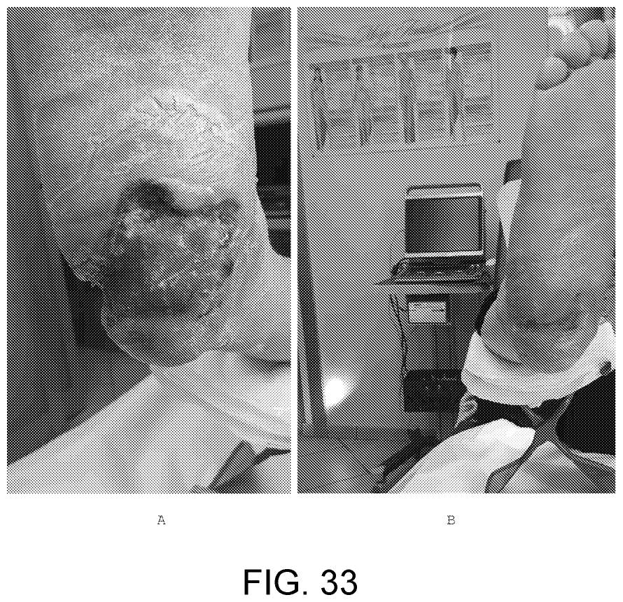

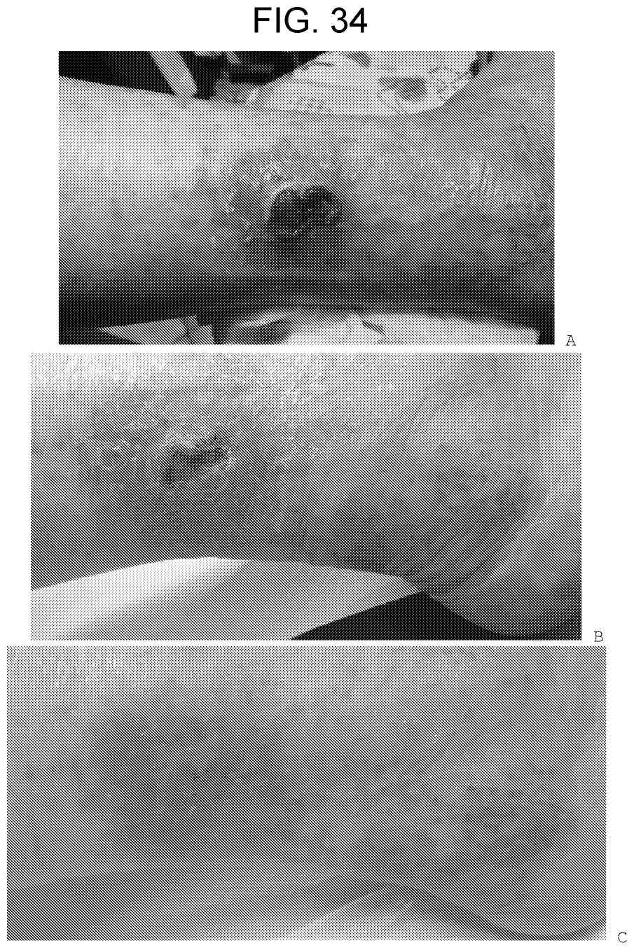

In most cases the healing times were much lower than those observed in the control group treated with classical drug therapy and furthermore all the subjects experienced complete recovery and in no case was amputation of the affected limb necessary [FIGS. 32-33-34, before (A) and after (B and C) the therapy with the composition of the present patent].

e) To evaluate the effectiveness of the preparations of the present invention in ophthalmology, the clinical trials performed concerned the treatment of persistent corneal epithelial defects, corneal surgery, and the treatment of dry eye syndrome. As regards the treatment of post laser-assisted in situ ocular surface keratomileusis or Laser-ASsisted In situ Keratomileusis—LASIK and the treatment of chronic ocular hypotonia after glaucoma surgery, the efficacy of the preparations of the present invention is demonstrated by the previous experiments.

Clinical trials were conducted on humans for keratoconjunctivitis sicca (dry eye syndrome) and lesions connected thereto (abrasions and corneal ulcers), and on dogs for abrasions and corneal ulcers of a traumatic, chemical, infectious, metabolic nature, etc., comprising those resulting from persistent corneal epithelial defects which do not tend to heal (limbal stem cell deficiency or LSCD).

The experimentation on the dog served not only to test the object of the present invention in veterinary medicine, but also to verify the effectiveness thereof in human medicine, since the dog, due to the shape and size of the cornea, represents the ideal animal model for similar human corneal diseases.

For the human trial, a total of 30 patients with keratoconjunctivitis sicca (dry eye syndrome) were divided into three homogeneous groups (A, B and C) of 10 patients each. One of the three groups (A) was treated with the composition referred to as the “Topical Formulation A” (shown in Example 1) in the form of eye drops, the second group (B) was treated with the same eye drops containing, however, only the colostrum isolate mixture and hyaluronic acid as the active ingredient, the third group (C) was treated with the same eye drops containing, however, only APCs and hyaluronic acid as the active ingredient (for all formulations the therapy consisted of three daily administrations, by conjunctival instillation, for 10 consecutive days).

The pathological conditions of the cornea, pre- and post-treatment, were evaluated using the guidelines recommended by the National Eye Institute/Industry (NEI), a government body dependent on the National Institutes of Health (NIH)—USA.

The NEI-recommended scoring system divides the cornea into five areas (central, upper, temporal, nasal and lower) and for each area, the severity of corneal fluorescein staining is graded on a scale of 0 to 3 based on the reference figures. Therefore, the maximum score is 15.

For each patient, in the case of bilateral lesions, the eye with the highest NEI score was considered.

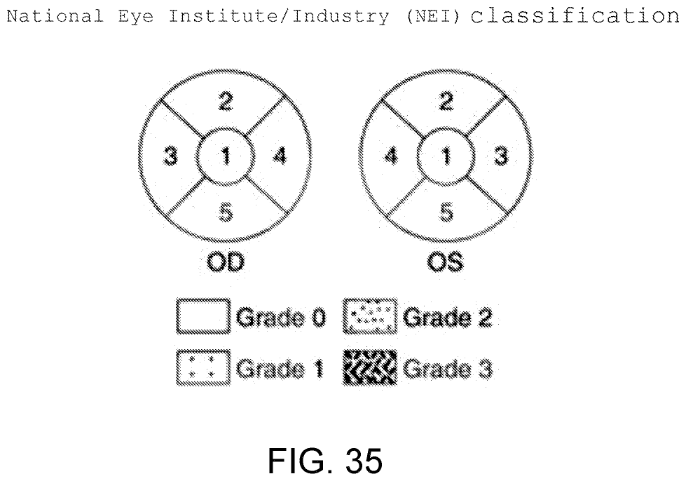

FIG. 35 shows the graph, which allows assigning the score of each of the 5 cornea zones and the total score based on the comparison with the reference images.

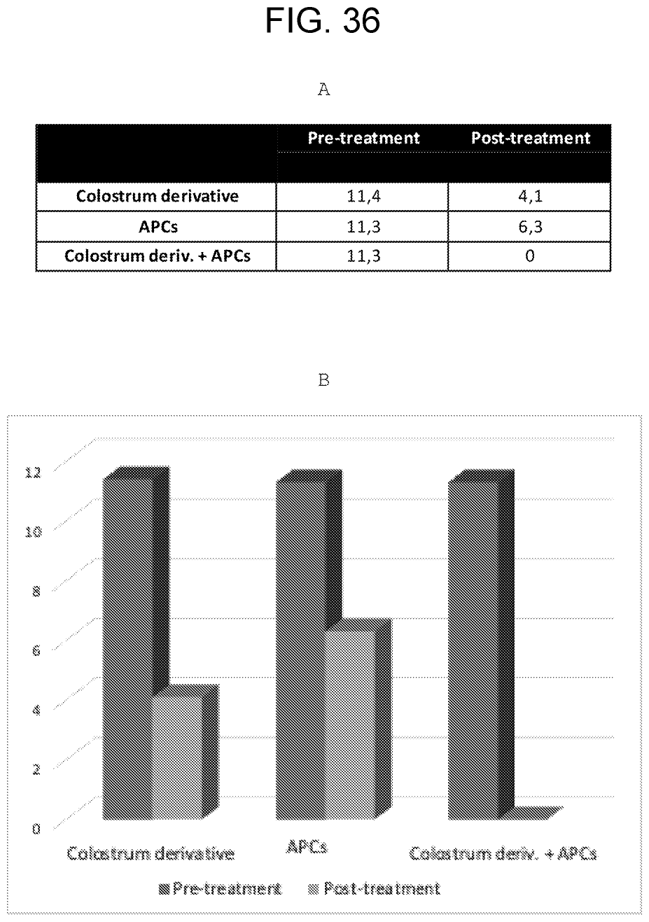

The table of FIG. 36A shows the average of the scores assigned to each patient, before and after the relative treatment with one of the three tested products (colostrum isolate mixture, APCs, colostrum isolate mixture+APCs). The same data are plotted in FIG. 36B.

The statistical calculations also show that the difference in the post-treatment averages is significant (p<0.05) among all the products tested. This demonstrates the greater effectiveness of the colostrum isolate mixture+APCs of the present invention, with respect to all the other reference products.

For the dog trial, a total of 30 subjects suffering from corneal abrasions and ulcers of a traumatic, chemical, infectious, metabolic nature or resulting from persistent corneal epithelial defects which do not tend to heal (limbal stem cell deficiency or LSCD), were divided into three homogeneous groups of 10 subjects each and treated according to the same therapeutic scheme used for the clinical trials on humans.

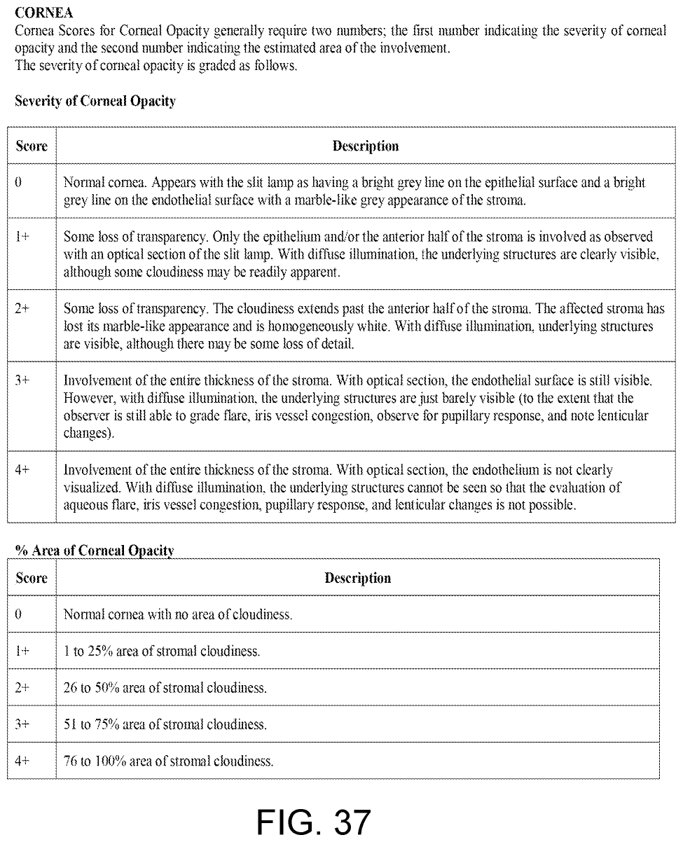

In this case, the Modified Hackett—Mcdonald Scoring Scale shown in FIG. 37 was used to assess the pathological conditions of the cornea, pre- and post-treatment.

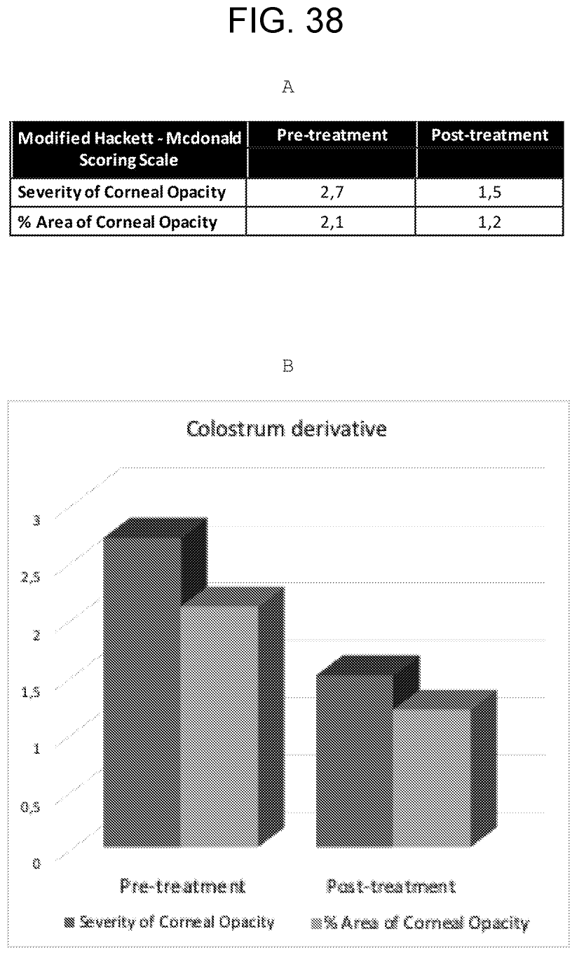

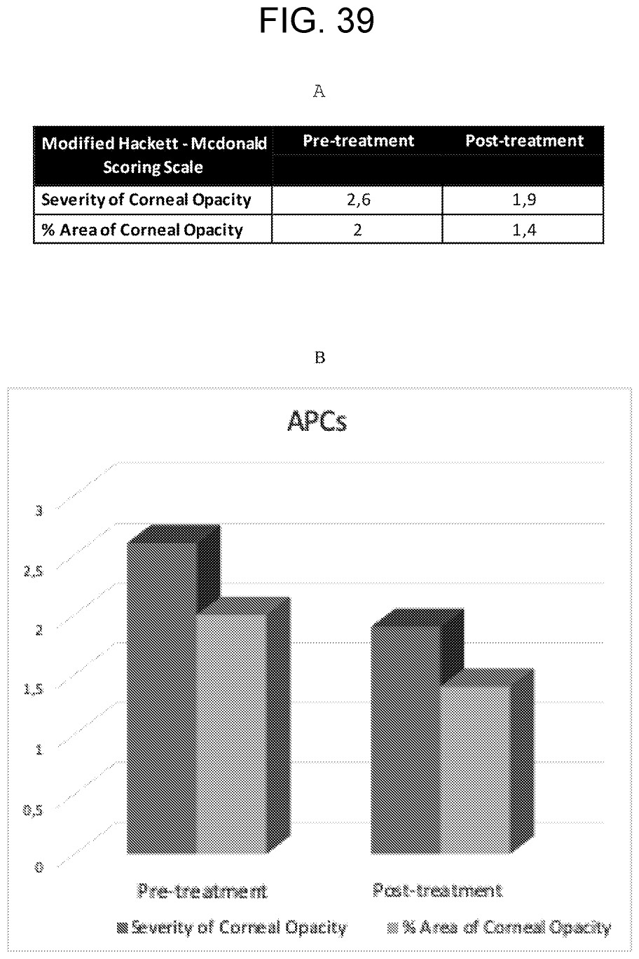

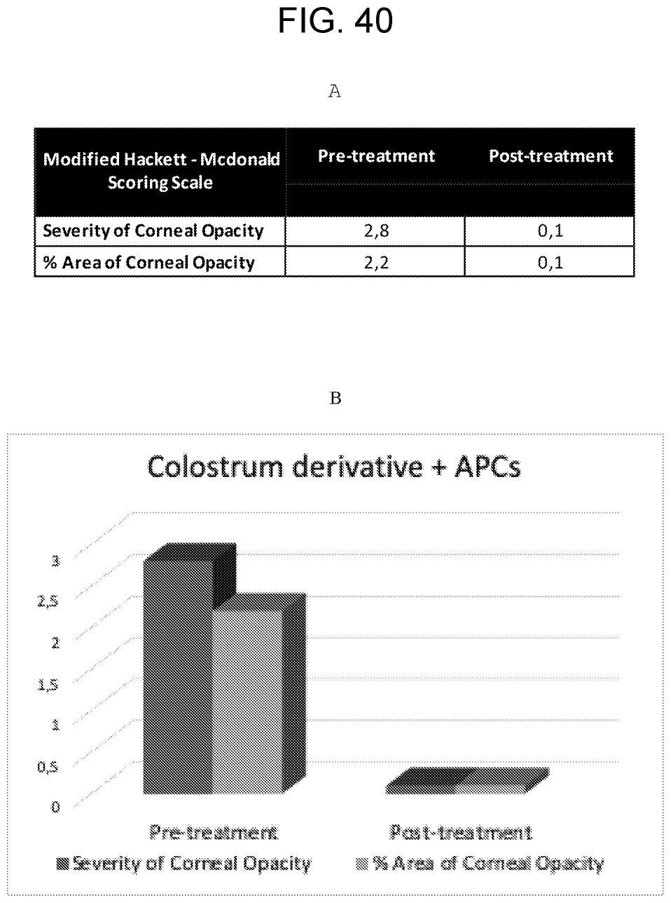

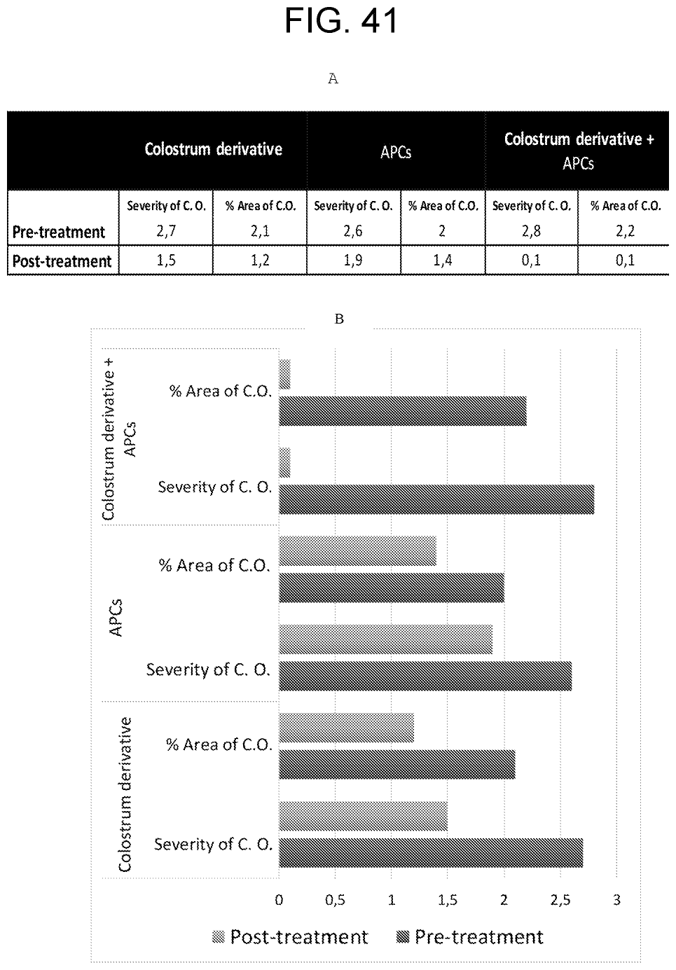

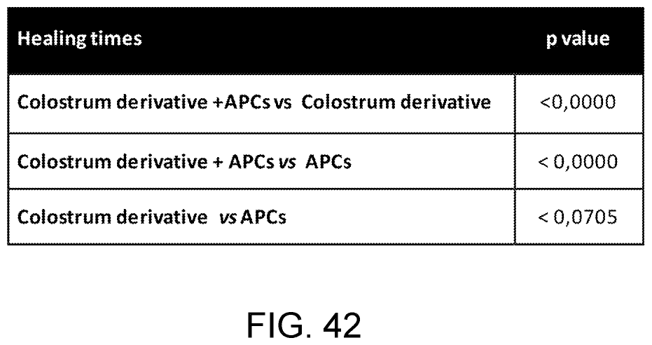

The tables of FIGS. 38A, 39A and 40A show the averages of the scores assigned to each subject, before and after the relative treatment with one of the three tested products (colostrum isolate mixture, APCs, colostrum isolate mixture+APCs). The same data are plotted in FIGS. 38B, 39B and 40B, while FIGS. 41A and 41B compare the results obtained in the clinical trials from each product tested and FIG. 42 reports the results of the statistical analysis.

Student's t (p<0.05) was used to compare the results obtained from the clinical trials. Also, in this case the data obtained show that the product containing the colostrum isolate mixture+APCs of the present invention, procured the greatest benefits with respect to the other products from the clinical perspective.

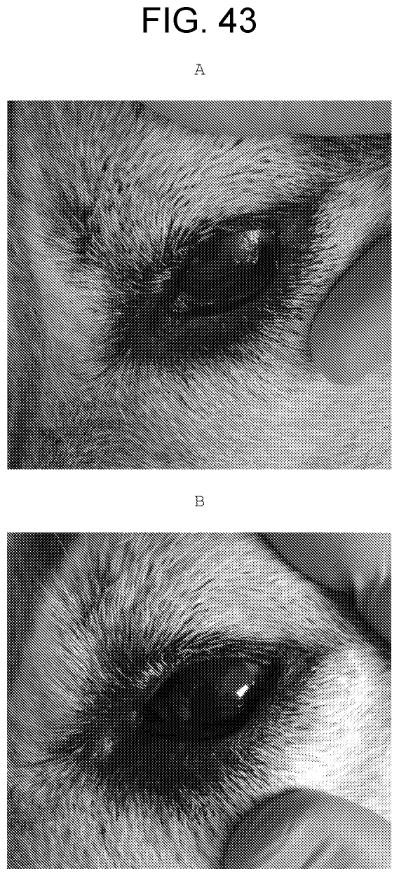

Therefore, the compound of the present invention, in both humans and in animals, has allowed obtaining the healing of corneal lesions with complete healing and negativization upon examination with fluorescein (FIG. 43: A before treatment, B after treatment with the composition of the present patent), accompanied by the disappearance of clinical symptoms (blepharospasm, photophobia, epiphora). It is also important to underline that in the case of ulcers and corneal abrasions, in none of the patients/subjects treated with the colostrum isolate mixture+APCs were there residual scars or permanent corneal leukomas, outcomes which are very frequently observed in all corneal lesions, even superficial, subjected exclusively to classic drug therapy. Furthermore, the local treatment with the product of the present invention did not give rise to any side effects, thus proving not only effective but also safe.

In all the clinical trials carried out, the results obtained with the composition based on autologous platelet concentrates and a colostrum isolate mixture of biological factors described in the present patent application were far better than the results obtained with all the other products tested, both in terms of effectiveness and healing speed.

Student's t (p<0.05 significant values) was used to compare the results obtained from the clinical trials. The statistical calculations show that the difference in post-treatment averages is significant (p<0.05) or highly significant (p<0.01) in all the clinical trials performed.

The above demonstrates the greater effectiveness of the composition based on platelet concentrates, preferably autologous, and of a colostrum isolate mixture of biological factors of the present invention, with respect to all the other products.

In particular, it has been surprisingly found that by combining APCs with bioactive factors derived from colostrum, the correct growth and regeneration of tissues is modulated and essential nourishment is provided to cells in the growth and proliferation phase.

From the above description of the present invention, the advantages given by the suggested formulations will be immediately apparent.

In particular, it was observed how the effectiveness of these formulations is closely linked to the synergy of the two components and the functional interdependence thereof.

In fact, the bioactive substances extracted from the colostrum provide nourishment to the cells in the growth and proliferation phase and provide further bioactive factors capable of modulating the action of the growth factors already present in the APCs.

Only by virtue of this synergy can the growth factors act, promoting the reparative process; this occurs by stimulating neoangiogenesis, even in poorly vascularized areas, and the regeneration of damaged tissues, which are replaced with cells of the same type, thus avoiding the replacement thereof with connective tissue (fibrosis). Furthermore, the application of the compositions of the invention prevents the onset of hypertrophic or keloid scars, which represent one of the most common wound healing disorders.

The clinical tests carried out have shown a considerably higher activity of the compositions described herein with respect to a product comprising only the portion based on autologous platelet concentrates or only the portion derived from colostrum.

Furthermore, the treatment with the compositions of the present invention did not give rise to any side effects, thus proving not only effective but also safe.

In order to meet contingent and specific needs, those skilled in the art can make adaptations and modifications to the present invention described above, and can replace components with other similar ones, without however departing from the scope of the claims set forth below.

For example, one or more excipients can be modified according to the pharmaceutical form selected.

1. A composition comprising platelet concentrates and a colostrum isolate mixture rich in biological factors, wherein the composition is deprived of immunoglobulins IgG, IgA, and IgM.

2. The composition of claim 1, wherein the platelet concentrates have a platelet concentration of about 1×10⁶/μL±20%.

3. The composition of claim 1, comprising wherein the composition comprises said colostrum isolate mixture rich in biological factors in an amount of about 10-1000 mg.

4. The composition of claim 1, wherein said platelet concentrates are represented by: platelet-rich plasma (PRP), PRP rich in leukocytes (L-PRP), platelet-rich fibrin (PRF), and plasma rich in growth factors (PRGF).

5. The composition of claim 1, wherein said platelet concentrates are autologous platelet concentrates (APCs).

6. The composition of claim 1, wherein the platelet concentrates are activated or non-activated.

7. The composition of claim 1, wherein said colostrum isolate mixture rich in biological factors is isolated from human or animal colostrum.

8. The composition of claim 1, wherein said colostrum isolate mixture rich in biological factors is isolated from colostrum of dairy cows.

9. The composition of claim 8, wherein said colostrum isolate mixture rich in biological factors is isolated from colostrum collected before a newborn has had the opportunity to nurse and empty an udder of the first fraction produced, regardless of time elapsed since the moment of birth, at the second or third birth.

10. The composition of claim 1, wherein said colostrum isolate mixture rich in biological factors comprises growth factors, cytokines, chemotactic factors, stem cell stimulating factors, complement proteins, antibacterial and antiviral factors.

11. The composition of claim 1, wherein the colostrum isolate mixture is included in a liposomal formulation.

12. The composition of claim 1, further comprising one or more of the following compounds: hyaluronic acid, amino acids, hydroxyapatite, β-tricalcium phosphate.

13. The composition of claim 1, wherein the composition is a topical composition or an injection composition.

14. The composition claim 13, wherein, in 5 mL of plasma, the topical composition has the following formulation:
APCs 1×10⁶/μL, understood as the number of platelets,
colostrum isolate mixture 500 mg, and
hyaluronic acid 125 mg.

15. The topical composition claim 14, wherein, in 5 mL of plasma, the topical composition further comprises, alternatively:
hydroxyapatite q.s.,
β-tricalcium phosphate 5 g.

16. The composition of claim 13, wherein, in 5 mL of plasma, the injection composition has the following formulation:
APCs 1×10⁶/μL, understood as the number of platelets,
colostrum isolate mixture 50 mg, and
hyaluronic acid 125 mg.

17. The composition claim 16, wherein, in 5 mL of plasma, the injection composition further comprises:
amino acid mixture 100 mg, and
hydroxyapatite (40% microspheres gel) 1 mL.

18. A topical or injection formulation comprising the composition of claim 1, in a form selected from the group consisting of: liquid solutions, lotions, foams, sprays, creams, salves, ointments, pastes, gels, membranes, clots, powders or other pharmaceutically suitable forms.

19. (canceled)

20. A method for treating conditions requiring tissue repair and regeneration, in humans or animals, the method comprising administering to said humans or animals the composition of claim 1.

21. The method of claim 20, wherein the conditions requiring tissue repair and regeneration, in humans or animals, comprise oral and maxillofacial regenerative surgical procedures comprising conditions selected from the group consisting of: alveolar recess after tooth extraction, and periodontal defects.

22. The method of claim 20, wherein the conditions requiring tissue repair and regeneration, in humans or animals, comprise orthopedics and sports medicine treatment of conditions selected from the group consisting of tendon injuries, osteoarthritis, degenerative disease of intervertebral discs, of muscle injuries, fractures and nonunions.

23. The method of claim 20, wherein the conditions requiring tissue repair and regeneration, in humans or animals, comprise dermatology and aesthetic medicine, treatment of conditions selected from the group consisting of vitiligo, acne, alopecia, stretch marks, skin atrophy, and vaginal atrophy.

24. The method of claim 20, wherein the conditions requiring tissue repair and regeneration, in humans or animals, comprise plastic and reconstructive surgery, treatment of conditions selected from the group consisting of burns, wounds, skin ulcers, keloids and hypertrophic scars, and soft breast tissue defects.

25. The method of claim 20, wherein the conditions requiring tissue repair and regeneration, in humans or animals, comprise ophthalmology, treatment of conditions selected from the group consisting of persistent corneal epithelial defects, dry eye syndrome, post laser-assisted in situ keratomileusis (LASIK) ocular surface syndrome (OSS), and chronic ocular hypotonia after glaucoma surgery.
